SEQUENCE LISTING (14 sequences)

SEQ ID NO: 1 - DNA, 14 bp, Synthetic construct
  source 1..14: mol_type = other DNA
gcgaacagtcaccc

SEQ ID NO: 2 - DNA, 14 bp, Synthetic construct
  source 1..14: mol_type = other DNA
gggtgactgttcgc

SEQ ID NO: 3 - DNA, 14 bp, Synthetic construct
  source 1..14: mol_type = other DNA
gctccgaaccacag

SEQ ID NO: 4 - DNA, 14 bp, Synthetic construct
  source 1..14: mol_type = other DNA
ctgtggttcggagc

SEQ ID NO: 5 - protein, 4 aa, Synthetic construct
WRPW

SEQ ID NO: 6 - protein, 19 aa, Synthetic construct
RLITLADHICQIITQDFAR

SEQ ID NO: 7 - protein, 25 aa, Synthetic construct
QINDLYSTDRPESAEAPDLQSWELR

SEQ ID NO: 8 - protein, 27 aa, Synthetic construct
ELQKSIGHKPEPTEEWELIKTVTEAHV

SEQ ID NO: 9 - protein, 18 aa, Synthetic construct
STPSSKTKDLGHNDKKSS

SEQ ID NO: 10 - DNA, 20 bp, Synthetic construct
  source 1..20: mol_type = other DNA
actttgcaaggagagccaaa

SEQ ID NO: 11 - DNA, 20 bp, Synthetic construct
  source 1..20: mol_type = other DNA
gctatcaccaggctcggtag

SEQ ID NO: 12 - DNA, 20 bp, Synthetic construct
  source 1..20: mol_type = other DNA
gggttagcgatggaggtagg

SEQ ID NO: 13 - DNA, 20 bp, Synthetic construct
  source 1..20: mol_type = other DNA
gatcctcccgaatcaaccct

SEQ ID NO: 14 - protein, 29 aa, Synthetic construct
SGLMDLDFDDLADSGLMDLDFDDLADSGC

(19) United States
(12) Patent Application Publication
(10) Pub. No.: US 2025/0170269 A1
(43) Pub. Date: May 29, 2025

(54) NANOPARTICLES FOR DIRECT REGULATION OF MITOCHONDRIAL GENE EXPRESSION

(71) Applicant: Rutgers, The State University of New Jersey, New Brunswick, NJ (US)

(72) Inventors: Ki-Bum Lee, Monmouth Junction, NJ (US); Letao Yang, Piscataway, NJ (US)

(73) Assignee: Rutgers, The State University of New Jersey, New Brunswick, NJ (US)

(21) Appl. No.: 18/910,825

(22) Filed: Oct. 9, 2024

Related U.S. Application Data
(60) Provisional application No. 63/588,864, filed on Oct. 9, 2023.

(51) Int. Cl. A61K 47/69; B82Y 5/00; C12N 15/87

(52) U.S. Cl. CPC A61K 47/6923; A61K 47/6935; C12N 15/87; B82Y 5/00

(57) ABSTRACT

Compositions comprising biologically active synthetic nanoparticle constructs and methods of use thereof to modify mitochondrial gene expression including transcriptional repression and transcriptional activation.

### CROSS-REFERENCE TO RELATED APPLICATIONS

This application claims the benefit under 35 U.S.C. § 119(e) of the earlier filing date of U.S. Provisional Application No. 63/588,864, filed on Oct. 9, 2023, which is hereby incorporated by reference in its entirety.

### STATEMENT REGARDING FEDERALLY SPONSORED RESEARCH

This invention was made with government support under grant number CBET-1803517 awarded by the National Science Foundation (NSF), grant numbers CSCR17IRG010 and CSCR16ERG019 awarded by the New Jersey Commission on Spinal Cord Research Award, and grant numbers R21AR071101, 1R01DC016612, 3R01DC016612-01S1, and 5R01DC016612-02S awarded by the National Institutes of Health (NIH). The U.S. Government has certain rights in this invention.

### REFERENCE TO AN ELECTRONIC SEQUENCE LISTING

The contents of the electronic sequence listing (070439_01849SeqList.xml; Size: 13,321 bytes; and Date of Creation: Sep. 24, 2024) is herein incorporated by reference in its entirety.

### FIELD OF THE INVENTION

This invention relates to biologically active synthetic nanoparticle construct compositions and methods of their use in regulating, mediating, or modifying biological activity and processes including gene expression and the cellular processes that rely on mitochondrial DNA.

### BACKGROUND OF THE INVENTION

Abnormal mitochondrial DNA (mtDNA) transcription is associated with a multitude of human diseases. Consequently, effective and innovative methods of site-specifically regulate mitochondrial DNA (mtDNA) transcription are required for both investigating and treating these disorders. However, it is difficult to deliver DNA binding motifs, such as DNAs, RNAs, and small molecules, into the targeted mitochondria both efficiently and selectively.

Multiple copies of mtDNA exist in mammalian cells in the heteroplasmic form, comprising both wild-type alleles and mutations. Current approaches to mtDNA transcription manipulation have primarily relied on exogenous delivery of transcription factors (e.g., Mitochondrial transcription factor A (TFAM)) via DNA-base editing nucleases such as transcription activator-like effector nucleases (TALENs), CRISPR-Cas9 systems, and DddA (double-stranded DNA deaminase A)-derived cytosine base editors (DdCBEs). These methods can potentially bind to mutant mtDNA and trigger transcriptional repression or base-editing to reduce the pathogenic burden while leaving wild-type alleles alone. Additionally, it is difficult to deliver these systems into the cytoplasm and mitochondrial to treat for mitochondria-associated diseases. This is due to the fact that most mitochondrial transcription factors and base editing tools encounter significant barriers during intracellular delivery or circulation in the blood. Additionally, due to their enormous size, their mobility is hampered when passing through cellular membranes and mitochondrial membranes, which poses an additional obstacle to achieving maximum efficiency in mitochondrial transcription regulation. Small-molecule-based approaches to mimic the function of mitochondrial transcription factors (TFs) have been demonstrated, albeit with limited effectiveness due to their poor solubility and delivery efficiency into targeted mitochondria.

In response to the challenges outlined above, nanoparticle (NP)-mediated drug delivery systems (DDSs) may be used for developing novel therapeutic interventions for mitochondria-associated diseases such as cancer and neurological diseases. When binding to mitochondria-associated biomolecules, NP-mediated DDSs can increase drug solubility, cellular targeting specificity, and multivalency effects. Several NP-based DDSs have demonstrated the capacity to significantly impact mitochondrial activity by altering the cellular redox environment, increasing the temperature of the cytoplasm, and delivering medicines that target mitochondrial pathways. Nevertheless, their potential for direct, effective, and target-specific modulation of mitochondrial gene expression is largely unexplored.

The mitochondrial genome of mammals encodes 13 proteins. Because each of these proteins has a distinct function in mitochondria-mediated disease modeling and therapy, the ability to selectively regulate each mitochondrial gene transcription may facilitate improved treatment of mitochondria-associated diseases. Consequently, there remains a long-felt need to develop an effective, systematic, and selective method to modulate mitochondrial gene expression levels by mimicking the functions and structures of mitochondrial TFs.

### SUMMARY OF THE INVENTION

The invention described herein involves biologically active synthetic nanoparticle constructs and methods of use of the biologically active synthetic nanoparticle constructs. Some embodiments of this invention comprise use of the biologically active synthetic nanoparticle constructs to modify mitochondrial gene expression, including transcription repression.

One aspect of the present invention provides a biologically active synthetic nanoparticle construct comprising:
i. a nanocluster that allows for conjugation of multiple biomolecular ligands,
ii. a plurality of linkers,
iii. a plurality of single copies of mtDNA-binding domains constructed to bind selectively to the light strand promoter or heavy strand promoter of a target mitochondrial gene and suppress gene expression; and
iv. a plurality of mitochondrial penetrating linkers,
wherein the nanoparticle selectively targets the mitochondria of a cell.

In one embodiment, the nanoparticle size is between 1 to 3 nm.

In one embodiment, the nanocluster is gold, platinum, silver, aluminum, copper or alloys thereof, or a chalcogen. In a further embodiment, the nanocluster is gold. In a further embodiment, the nanocluster exhibits innate near-infrared red (NIR) fluorescence.

In one embodiment, the linkers are selected from the group consisting of hydrophilic glutathione (GSH) peptides, proteins, polyethylene glycol, polysaccharides, lipids, alkynes, alkanes, polyamines, carbene-terminated ligands, and zwitterionic polymers. In a further embodiment, the linkers are GSH peptides.

In one embodiment, the mitochondrial penetrating linkers are selected from the group consisting of triphenylphosphonium, phenylalanine- and cyclohexyl alanine-based mitochondrial penetrating peptides.

In one embodiment, the mtDNA-binding domains are pyrrole-imidazole polyamide (PIP) ligands. In a further embodiment, the PIP ligands are LSP-NH₂ PIP ligands or HSP-NH₂ PIP ligands.

In one embodiment, the conjugation ratio between mitochondrial penetrating linkers and PIPs is between 1:1 and 1:4.

In one embodiment, the nanoparticle inhibits mitochondrial gene expression. In one embodiment, the mitochondrial gene is selected from the group consisting of ATPF06, ATPF08, COI, COII, COIII, CYTB, ND1, ND2, ND3, ND4, ND4l, ND5, and ND6.

In a further embodiment, inhibiting mitochondrial gene expression increases ROS levels.

### BRIEF DESCRIPTION OF THE DRAWINGS

FIGS. 1A-1C are schematic diagrams illustrating biologically active synthetic nanoparticle construct-based regulation of mitochondrial gene transcription. FIG. 1A depicts the overall design of the biologically active synthetic nanoparticle construct. Inset image (200×200 μm) in the lower panel is cellular imaging of Hela cells from the fluorescence of biologically active synthetic nanoparticle constructs. FIG. 1B shows the delivery of biologically active synthetic nanoparticle constructs into mitochondria for targeting ND6 (NADH-ubiquinone oxidoreductase chain 6) gene in mitochondria DNA. The schematic diagram shows the human mitochondrial genome and the box indicates the location of ND6 gene in the light strand promoter region. FIG. 1C shows illustrations of ND6 mtDNA suppression-induced ROS activation. Inset images (200×200 μm) are dichlorodihydrofluorescein diacetate-based staining of intracellular ROS. HeLa cells were treated by biologically active synthetic nanoparticle constructs followed by imaging with 2′-7′-Dichlorodihydrofluorescein diacetate (DCFHDA)-based detection of ROS.

FIGS. 2A-2G show the synthesis of biologically active synthetic nanoparticle constructs for mitochondria targeting. FIG. 2A is a schematic diagram illustrating conjugation of different domains onto glutathione (GSH)-functionalized Au nanocluster to form the biologically active synthetic nanoparticle constructs. EDC/NHS (1-Ethyl-3-(3-dimethylaminopropyl) carbodiimide/N-hydroxysuccinimide) coupling between the carboxylic group in the GSH and the primary amine group in the PIP and MPP (mitochondria-penetrating peptide) was used for the conjugation. FIG. 2B shows the chemical structures of different domains used in the synthesis of the biologically active synthetic nanoparticle construct. FIGS. 2C and 2D depict transmission electron microscope (TEM) (FIG. 2C) data and quantification of TEM sizes of nanoclusters (FIG. 2D). FIG. 2E is a graph of zeta potential measurement confirming the conjugation of cationic/neutrally charged MPP and PIP ligands to the anionic nanoclusters which leads to a decrease of negative charge on the nanocluster surface. FIG. 2F is a graph of fluorescence microscope data of the constructed biologically active synthetic nanoparticle constructs. The inset image is showing the fluorescence from nanocluster aqueous in solution under exposure of UV (λ=325 nm). FIG. 2G depicts confocal microscope characterization of biologically active synthetic nanoparticle construct-based selective targeting of mitochondria.

FIG. 3 illustrates representative cell fluorescent images after HeLa cells were treated with gold nanocluster and their biocompatibility. The minimum fluorescence indicates the minimal cellular uptake of the nanocluster alone, highlighting the importance of conjugation of MPP and LSP (light strand promoter) in the biologically active synthetic nanoparticle construct design.

FIGS. 4A-4E show the comparison of biocompatibility of biologically active synthetic nanoparticle constructs and PIP molecules. Adult stem cells, monocytes, iPSCs, and HeLa cells were treated with biologically active synthetic nanoparticle constructs or the corresponding PIP molecules for 48 hours before being analyzed by PrestoBlue™ Cell Viability Assay. FIG. 4A is a graph of HeLa cell viability. FIG. 4B is a graph of iPSC viability. FIG. 4C is a graph of iPSC viability. FIG. 4D is a graph of monocyte viability. FIG. 4E is a graph of adult stem cell viability.

FIGS. 5A-5E show biologically active synthetic nanoparticle construct-based mitochondrial gene regulation. FIG. 5A is a schematic diagram showing the working principle of biologically active synthetic nanoparticle constructs for mitochondrial gene regulation. FIG. 5B depicts the experimental design (timeline and control groups) of in vitro validation of the biologically active synthetic nanoparticle construct-based regulation of mtDNA transcription. FIG. 5C is a schematic diagram showing the in vitro model for studying gene transcription in mitochondria based on ND6 mRNA level and using 16S gene as a control gene. FIG. 5D is a graph of selective suppression of ND6 by LSP-targeting biologically active synthetic nanoparticle constructs. n=3 biological replicates. *P<0.05 by one-way ANOVA test with Tukey post-hoc analysis. FIG. 5E is a graph of the optimization of biologically active synthetic nanoparticle constructs by modulating the conjugation ratio between MPP and PIP from 1:1, 1:2, to 1:4, and treating cells with different concentrations of biologically active synthetic nanoparticle constructs of ND6 by LSP-targeting biologically active synthetic nanoparticle constructs. n=3 biological replicates.

FIG. 6 illustrates mitochondrial gene regulation by a biologically active synthetic nanoparticle construct in a hiPSC-NSC (human induced pluripotent stem cell-derived neural stem cell). Biologically active synthetic nanoparticle construct-based mitochondrial gene regulation was reproduced in a completely different (hiPSC-NSC line by using identical procedures (other than no OptiMEM® media used) used for HeLa cells. n=3 biological replicates.

FIG. 7 shows the size-dependent effects of a biologically active synthetic nanoparticle construct-based modulation of mitochondrial gene expression. The four images on the left panel are representative TEM characterization of biologically active synthetic nanoparticle constructs constructed from 10, 5, 3, and 2 nm gold nanoparticle/nanoclusters, respectively. 10 and 5 nm gold nanoparticle were initially citrate-capped followed by glutathione ligand exchange.

FIGS. 8A-8C show that biologically active synthetic nanoparticle construct-mediated ND6 suppression leads to intracellular ROS activation. FIG. 8A is a schematic diagram showing that biologically active synthetic nanoparticle construct-mediated ND6 suppression leads to alteration of cellular redox status. FIG. 8B is a graph showing the selective and concentration-dependent activation of ROS by biologically active synthetic nanoparticle constructs targeting LSP. FIG. 8C shows representative ROS staining quantified in FIG. 8B (from DCF assay) showing the selective and concentration-dependent activation of ROS by biologically active synthetic nanoparticle constructs targeting LSP. Value shown in FIG. 8B indicates the concentration of biologically active synthetic nanoparticle constructs or nanoclusters (unit: ug/mL). n=3 biological replicates.

FIG. 9 shows biologically active synthetic nanoparticle construct size-dependent effects on mitochondria ROS activation. The fluorescence of DCFDA (dichlorodihydrofluorescein diacetate) was used as an indicator for intracellular ROS in HeLa cells.

### DETAILED DESCRIPTION OF THE INVENTION

### 1. Overview

Unless otherwise defined, all technical and scientific terms used herein have the same meaning as commonly understood by one of ordinary skill in the art to which this invention pertains. In the case of conflict, the present document, including definitions will be controlled. This invention concerns biologically active synthetic nanoparticle constructs and their methods of use. Generally, the biologically active synthetic nanoparticle constructs are comprised of a nanocluster that allows for conjugation of multiple biomolecular ligands, a plurality of linkers, a plurality of single copies of mtDNA (mitochondrial DNA)-binding domains constructed to bind selectively to the light strand promoter or the heavy strand promoter of a target mitochondrial gene and suppress gene expression, and a plurality of mitochondrial penetrating linkers (e.g., mitochondria-penetrating peptides (MPPs); MPPs are a class of mitochondrial penetrating peptides), wherein the nanoparticle selectively targets the mitochondria of a cell. The nanoparticle constructs may be used, for example, to enable significantly repress mitochondrial gene expression in cells to treat mitochondrial-associated diseases or disorders (See Section 3E below). Furthermore, the nanoparticle constructs may be combined with a delivery vehicle for effective delivery into a host system (e.g., eukaryotic host).

As used herein, the term “light strand promoter” (LSP) and “heavy strand promoter” (HSP) refers to two different promoters located in opposite DNA strands that are involved in the transcription of human mitochondrial DNA, which consists of light and heavy strands. Human mitochondrial genome transcription is bi-directional and regulated by these two closely spaced promoters. Both the LSP and HSP are located within a non-coding region.

As used herein, the term “nanocluster” refers to a group of nanoparticles that have at least one nanoscale dimension and size distribution.

As used herein, the terms “inhibits mitochondrial gene expression” or “inhibition of mitochondrial gene expression” refers to complete or partial inhibition of mitochondrial gene expression.

As used herein, the term “transcriptional repression” refers to complete or partial inhibition of mitochondrial genes.

As used herein and in the appended claims, the singular forms “a,” “and” and “the” include plural references unless the context clearly dictates otherwise.

All publications mentioned herein are incorporated herein by reference in their entireties.

### 2. Biologically Active Synthetic Nanoparticle Construct

The present invention provides biologically active synthetic nanoparticle constructs functionalized with both mtDNA-binding domains and mitochondrial penetrating linkers, and optionally other components. The biologically active synthetic nanoparticle constructs are capable of repressing transcriptional activity, and therefore capable of regulating gene expression in living cells in a non-viral manner. These biologically active synthetic nanoparticle constructs have a small size and multifunctional properties to accommodate different types of biomolecules on a single nanoparticle, can permeate the mitochondrial membrane, have enhanced localization within the mitochondria while remaining intact, can initiate strong transcriptional repression in human induced pluripotent stem cell-derived neural stem cells (hiPSC-NSCs) and HeLa cells, and are nontoxic to cells under the condition studied. The biologically active synthetic nanoparticle constructs can bind to select sequences on mtDNA in hiPSC-NSCs and HeLa cells. In particular, biologically active synthetic nanoparticle constructs have a size between 1 to 3 nm such as 1 nm, 1.5 nm, 2 nm, 2.5 nm, 3 nm or any size in-between. This is advantageous compared to typical mitochondrial transcription factors that due to their enormous size, have impaired mobility when crossing mitochondrial membranes. The versatile and tunable properties of biologically active synthetic nanoparticle construct establish this as an effective platform for applications that require mitochondrial gene regulation such as oxidative phosphorylation and the generation of reactive oxygen species (ROS).

The nanocluster of the biologically active synthetic nanoparticle constructs provide a base platform upon which the other elements of the biologically active synthetic nanoparticle construct are bound to. There are many types of nanoclusters that that are appropriate for the present invention. For example, gold nanoparticles (AuNPs) are particularly effective as AuNPs are easy to synthesize, easy to handle, are FDA approved, and have multifunctional surfaces. However, there are many other substrates that are embodied by this invention. Other types of nanoparticles, for example but not limited to, platinum-based nanoparticles, magnetic nanoparticles, magnetic-core shell nanoparticles, silica nanoparticles, mesoporous nanoparticles, quantum dots, supramolecular nanoparticles, and polymer-based nanoparticles may all comprise the nanocluster in the present invention. In some embodiments, the nanocluster is gold, platinum, silver, aluminum, copper or alloys thereof, or a chalcogen. In further embodiments, the nanoclusters exhibit innate near-infrared (NIR) fluorescence.

The nanocluster of the biologically active synthetic nanoparticle constructs comprise a plurality of linkers that link the nanoparticles/nanoclusters to the mtDNA-binding domains and mitochondrial penetrating linkers (e.g., MPPs). In some embodiments, the linkers are selected from the group consisting of hydrophilic glutathione (GSH) peptides, proteins, polyethylene glycol, polysaccharides, lipids, alkynes, alkanes, polyamines, carbene-terminated ligands, and zwitterionic polymers. In some embodiments, the linkers are GSH peptides. In further embodiments, the GSH peptides have with two carboxylic groups for carbodiimide mediated coupling with amine-terminated pyrrole-imidazole polyamide (PIP) ligands and MPPs.

In some embodiments, the PIP ligands are LSP-NH₂ PIP ligands: ImPyPyβImPy-γ-PyPyβImPyPy-βDp-NH₂. In some embodiments, the PIP ligands are HSP-NH2 PIP ligands: ImImPy-γ-Py ImPyImPyPyPy-βDp-NH₂.

Mitochondrial DNA (mtDNA)-binding domains are those moieties which are capable of binding directly to target nucleotide sequences of interest on mtDNA, for example, but not limited to, a specific DNA sequence in a light strand promoter region or a heavy strand promoter region of a target gene. This invention utilizes mtDNA-binding domains for purposes of binding to mitochondrial nucleotide targets of interest to modulate mitochondrial gene transcription, for example, by repressing transcription, and thus affecting cell redox status, which modulates reactive oxygen species (ROS) generation. The mtDNA-binding domain is preferably comprised of a hairpin polyamide sequence motif comprised of N-methylpyrrole (Py) and N-methylimidazole (Im), as hairpin polyamide sequences exhibit high tunability and binding specificity as well as small molecular size. Hairpin polyamides function by binding to the minor groove of DNA through hydrogen bond interactions with a binding affinity comparable to naturally occurring DNA-binding proteins, as the Py and Im amino acids complement the A-T and G-C motifs on the DNA, respectively. In some embodiments, the minimum number of polyamide units is 10. The targeting selectivity is generally above 95% by the polyamide alone.

However, other mtDNA-binding domains are embodied by the present invention, and may include, for example but not limited to, zinc finger domains, triple forming oligonucleotides, transcription activator-like effectors, oligonucleotide analogs, locked-nucleic acids, and peptide nucleic acids. The sequences of representative mtDNA-binding domains are shown below in Table 1.

TABLE 1
mtDNA-binding domain sequences.

| SEQ ID NO | mtDNA-binding domain sequence |
|---|---|
| 1 | 5′-GCGAACAGTCACCC |
| 2 | 5′-GGGTGACTGTTCGC |
| 3 | 5′-GCTCCGAACCACAG |
| 4 | 5′-CTGTGGTTCGGAGC |

Given that the human mitochondrial genome was sequenced in 1981, one of ordinary skill in the art would understand how to design mtDNA-binding domains that can target mitochondrial genes of interest using standard methods known in the art.

Mitochondrial penetrating linkers include mitochondria-penetrating peptides (MPPs), which are synthetic cell-permeable peptides that are able to enter the mitochondria. MPPs can be cationic, but also lipophilic. The combination of these properties enables permeation of the hydrophobic mitochondrial membrane. Typically, it is difficult for molecules to cross the inner mitochondrial membrane (IMM), since the IMM limits diffusive transport. Examples of additional mitochondrial penetrating linkers include triphenylphosphonium, phenylalanine- and cyclohexyl alanine-based mitochondrial penetrating peptides. Examples of mitochondrial penetrating linkers are listed below in Table 2 and include phenylalanine-based mitochondrial penetrating peptides, triphenylphosphonium (TPP+)-based mitochondrial penetrating peptides, and cyclohexyl alanine-based mitochondrial penetrating peptide.

TABLE 2

Exemplary mitochondrial penetrating linkers.
Mitochondrial Penetrating Linkers
D-Arg-mtDNA-Orn-Phe
D-Orn-mtDNA-Orn-Phe
D-Arg-mtDNA-D-Arg-Phe

Note:
Orn indicates oligoribonucleotide.

The MPPs are linked to the nanoclusters of the biologically active synthetic nanoparticle constructs via covalent bonds (e.g., amide bonds). The short peptide linkers can be any linkers recognized by one of ordinary skill in the art.

The biologically active synthetic nanoparticle constructs may comprise further domains (conjugants) beyond the mtDNA-binding domains. These additional domains may be biologically active or inert; they may, for example but not limited to, facilitate stability of the nanoparticle construct, increase the safety of the nanoparticle construct, decrease toxicity, or they may interact with various cellular components. These additional domains may participate in transcriptional repression in a manner consistent with or independent of the mtDNA-binding domains. They may synergistically increase the efficiency or potency of the biologically active synthetic nanoparticle construct. These examples are not meant to be limiting but merely demonstrative of the possibilities of additional domains.

In some embodiments, the biologically active synthetic nanoparticle constructs may comprise a plurality of repression domains (RD). Repression domains (RD) are those domains that are capable of repressing transcriptional activity, for example, by activating repressor proteins. In the present invention, RDs are preferably, not but not necessarily, peptide sequences, and the peptide sequences are preferably, but not necessarily, synthesized in the D-isomer in order to resist intracellular degradation. A list of RDs that embodied by the present invention include but are not limited to those found in Table 3 below:

TABLE 3
Repression Domains

WRPW (SEQ ID NO: 5)
RLITLADHICQIITQDFAR (SEQ ID NO: 6)
QINDLYSTDRPESAEAPDLQSWELR (SEQ ID NO: 7)
ELQKSIGHKPEPTEEWELIKTVTEAHV (SEQ ID NO: 8)
STPSSKTKDLGHNDKKSS (SEQ ID NO: 9)

The addition of repression domains can be beneficial to synergistically enhance the suppression effects of the mtDNA-binding domains.

In some embodiments, the biologically active synthetic nanoparticle constructs may comprise a plurality of activation domains (AD). Activation domains (AD) are those domains that are capable of activating transcriptional activity, for example, by recruiting proteins such as RNAP and other factors that are required for transcriptional activation. ADs may be involved in triggering signaling cascades leading to expression of desired genes. In the present invention, ADs are preferably, but not necessarily, peptide sequences, and the peptide sequences are preferably, but not necessarily, synthesized in the D-isomer in order to resist intracellular degradation. Examples of an AD include TFAM and mtTFB (Mitochondrial Transcription Factor B). TFAM or mtTFB is required for mtDNA transcription and replication. One explicitly non-limiting example of an AD peptide domain is SGLMDLDFDDLADSGLMDLDFDDLADSGC (SEQ ID NO: 14). Other ADs are embodied by the present invention and may include, for example but not limited to, peptoids, amphipathic isoxasolidine, wrenchnolol, and amphipathic helix peptides.

The various domains of the biologically active synthetic nanoparticle construct, including mitochondrial localization domains (i.e., mtDNA-binding domains), or any other domains, are bound to a surface of the nanocluster. Preferably, the domains are bound covalently via a crosslinker, for example a crosslinker having a formula SH—R—COOH. The R group may be for example but not limited to, any derivative of an alkyl or alkoxy chain, straight chain, branched, or otherwise, so long as the chain is not too short, for example the backbone having less than 3 carbons in length, or too long, for example the backbone having greater than 200 carbons. For example, the R group may be comprised of polyethylene glycol (PEG) or undecanoic acid. However, one of ordinary skill in the art will recognize that there are many crosslinkers that are suitable for the purposes of this invention. Too short a chain risks aggregation, and too long a chain risks the overall size of the biologically active synthetic nanoparticle construct being unable to enter the nucleus due to size restrictions, thus rendering the construct ineffective. Preferably, the domain amino groups are coupled to crosslinker carboxylic acids via conventional EDC/NHS coupling, however one of ordinary skill in the art will recognize that there are many other chemical routes to conjugate the molecules, for example but not limited to, active ester coupling utilizing carbodiimides such as those reactions utilizing dicyclohexylcarbodiimide (DCC) and diisoproylcarbodiimide (DIC) or coupling reactions utilizing triazoles such as 1-hydroxy-benzotriazole (HOBt) and 1-hydroxy-7-aza-benzotriazole (HOAt).

In particular embodiments, the biologically active synthetic nanoparticle constructs are effectively uptaken by cells due to the zwitterionic nature of its surface ligands (anionic GSH linkers, cationic MPPs, and PIPs). Additionally, the endogenous fluorescence generated by quantum confinement at the nanoscale sizes of the nanoclusters can be applied to visualize cellular uptake and mitochondrial targeting.

### A. Regulation of Biologically Active Synthetic Nanoparticle Constructs

In some embodiments, the biologically active synthetic nanoparticle constructs can be further regulated in a reversible manner. In further embodiments, the nanoparticle constructs can be regulated by temperature, light, pH, and/or magnetic fields. Exposure to such conditions can cause dissociation of ligands and/or prevent ligand binding to the nanoparticle constructs.

In some embodiments, the biologically active synthetic nanoparticle constructs are stable for at least 24 hours following cellular uptake. In some embodiments, the biologically active synthetic nanoparticle constructs are secreted out of cells following a period of at least 24 hours.

In a further embodiment of the invention, the biologically active synthetic nanoparticle construct is at least one of biodegradable or biocompatible.

In yet another one embodiment of the invention, the ratio of mitochondrial penetrating linkers to the mtDNA-binding domains (e.g., PIPs) is tunable. In some specific embodiments, the conjugation ratio between mitochondrial penetrating linkers and PIPs is between 1:1 and 1:4 such as 1:1, 1:2, 1:3, and 1:4, or any ratio in-between.

### 3. Methods of Use of Biologically Active Synthetic Nanoparticle Constructs

### A. Transcriptional Repression

One aspect of the present invention is the use of biologically active synthetic nanoparticle constructs in order to repress transcription of particular target mitochondrial genes. The biologically active synthetic nanoparticle constructs as described herein are capable of repressing transcription. For methods of transcriptional repression, the biologically active synthetic nanoparticle constructs are comprised of a nanocluster, a plurality of linkers, a plurality of single copies of mtDNA-binding domains constructed to bind selectively to the light strand promoter or to the heavy strand promoter of a target mitochondrial gene and suppress gene expression, and a plurality of mitochondrial penetrating linkers, wherein the nanoparticle selectively targets the mitochondria of a cell. The mitochondrial penetrating linkers are essential because they enable transcriptional repression to occur within the mitochondria akin to how a nuclear localization signal directs a protein to enter the nucleus. Thus, the presently described biologically active synthetic nanoparticle constructs selectively modulate mitochondrial DNA transcription. Consequently, these biologically active synthetic nanoparticle constructs function as a transcription factor-mimetic nanoparticle with multiple domains integrated into a single platform for efficient and direct gene regulation within the mitochondria.

In some embodiments, the biologically active synthetic nanoparticle constructs repress any of the 13 mitochondrial genes: ATPF06, ATPF08, COI, COII, COIII, CYTB, ND1, ND2, ND3, ND4, ND4l, ND5, and ND6. In further embodiments, repression of mitochondrial genes results in increased ROS levels.

### B. Transcriptional Activation

One aspect of the present invention is the use of biologically active synthetic nanoparticle constructs in order to activate transcription of particular target mitochondrial genes. Endogenous genes are transcribed when transcriptional basal machinery, comprised of compounds including, but not limited to compounds such as general transcription factors (TFs), RNAP, SAGA, and mediators, are directed to a particular target gene sequence and thus, initiate transcription. The biologically active synthetic nanoparticle constructs as described in this invention are capable of activating mitochondrial transcription. For methods of transcriptional activation, the biologically active synthetic nanoparticle constructs are comprised of a nanocluster, a plurality of linkers, a plurality of single copies of mtDNA-binding domains, a plurality of mitochondrial penetrating linkers, and AD. When in this composition, the biologically active synthetic nanoparticle constructs mimic natural transcription factors, which are comprised of a plurality of single copies of mtDNA-binding domains, and AD. It is the presence of AD that distinguishes the biologically inactive synthetic nanoparticle constructs used for transcriptional activation from those used in transcriptional repression, as the ADs are involved in recruitment of the endogenous transcriptional basal machinery such as RNAP and mediators, thus initiating transcription. In one particular embodiment, the AD is TFAM or mtTFB. TFAM recruits mitochondrial RNA polymerase and transcription factor T2BM, which activates transcription. The plurality of mitochondrial penetrating linkers allows the biologically synthetic nanoparticle constructs to pass through the mitochondrial membrane, which is essential because transcription takes place in the mitochondria. The mtDNA-binding domains bind to the light strand promoter or heavy strand promoter of a target mitochondrial gene and the AD to recruit the components needed for transcriptional activation.

In some embodiments, the biologically active synthetic nanoparticle constructs activate any of the 13 mitochondrial genes: ATPF06, ATPF08, COI, COII, COIII, CYTB, ND1, ND2, ND3, ND4, ND4l, ND5, and ND6. In further embodiments, activation of mitochondrial genes results in decreased ROS levels.

### C. Vehicle Delivery Platform

Yet another aspect of the present invention is the use of a medical device that incorporates biologically active synthetic nanoparticle constructs. These biologically active synthetic nanoparticle constructs can be comprised of any combination of nanocluster, linking peptides, mtDNA-binding domains constructed to bind selectively to the light strand promoter or the heavy strand promoter of a target mitochondrial gene and suppress gene expression, and a plurality of mitochondrial penetrating linkers as disclosed in this invention.

A transport vehicle for implementation of the biologically active synthetic nanoparticle constructs may come in a variety of forms. For internal tissue use, the transport vehicle may optionally comprise an intravenous drip further including a saline solution and the cell differentiation medical device. The transport vehicle will recognize the importance of the size of an intravenous needle used and any container used in order to ensure free flow of the intravenous drip into the body.

For topical use, the transport vehicle may optionally include a biodegradable polymer scaffold.

The transport vehicle may further comprise biodegradable hydrogel for direct topical application of the biologically active synthetic nanoparticle constructs to a specific area of a surface of an organism. A hydrogel may significantly enhance the use of a growth media for cellular growth upon stimulation by the biologically active synthetic nanoparticle constructs.

The biologically active synthetic nanoparticle construct can be administered to a human subject via various modes of administration. Examples of such modes of administration include, but are not limited to, subcutaneous, topical, intravenous, internasal, intrathecal, intramuscular, intercranial injection, and systemic delivery.

### D. Treating Diseases (Such as Cancer or Neurodegenerative Diseases or Disorders)

Further aspects of the present invention include methods to treat a disease. In particular, the disease is a mitochondrial-associated disease or disorder. In further embodiments, the biologically active synthetic nanoparticle constructs as described above are used in a method to treat heteroplasmic mtDNA diseases. The method comprises administering biologically active synthetic nanoparticle constructs as described above to a human subject who has a disease. This embodiment may further comprise delivering the biologically active synthetic nanoparticle constructs to the subject via a medical device. In a further embodiment, the biologically active synthetic nanoparticle constructs may be administered in combination with another drug or therapeutic agent as discussed below in Section D.

In some embodiments, the disease is cancer. In some embodiments, the cancer is a mitochondrial-associated cancer. Mitochondria regulate cellular metabolism including oxidative phosphorylation, which can be either upregulated or downregulated in different cancer types. Dysregulated mitochondrial homeostasis can result in excess ROS levels that leads to DNA damage and cancer progression, inter alia. Furthermore, expression of mitochondria-encoded genes has been shown to be upregulated in various cancers. Thus, dysregulated mitochondrial gene expression contributes to cancer progression.

In some embodiments, the cancer includes, but is not limited to bladder cancer, brain cancer, breast cancer, bone cancer, colon cancer, endometrial cancer, esophageal cancer, head or neck cancer, kidney cancer, leukemia, lung cancer, lymphoma (such as non-Hodgkin's lymphoma and Hodgkin lymphoma), melanoma cancer, ovarian cancer, pediatric cancer, prostate cancer, rectal cancer, skin cancer, stomach cancer, testicular cancer, thyroid cancer, and environmentally induced cancers including those induced by asbestos, and any combinations thereof. In some embodiments, the cancer is early stage. In some embodiments, the cancer is metastatic. In some embodiments, the cancer is a resistant to previous lines of therapy. In further embodiments, the cancer is a secondary cancer due to a previously administered cancer therapeutic.

In preferred embodiments, the cancer is breast cancer, skin cancer, liver cancer, or pancreatic cancer.

In other embodiments, the disease is a neurodegenerative disease or disorder. In some embodiments, the neurodegenerative disease or disorder is a mitochondrial-associated neurodegenerative disease or disorder. Numerous studies have demonstrated that mitochondrial dysfunction is involved with the pathogenesis of neurodegenerative diseases including Parkinson's disease, Alzheimer's disease, and Huntington's disease, spinal cord injury, brain injury, ischemia, and stroke, among others. In these diseases mitochondria generally exhibit decreased activity of the respiratory chain enzyme and contain mutations in mtDNA. Neurodegenerative diseases include, but are not limited to Parkinson's diseases (PD), Alzheimer's (AD), Huntington's disease (HD), Friedreich's ataxia (FRDA), and amyotrophic lateral sclerosis (ALS).

In some embodiments, the disease is musculoskeletal disease. In some embodiments, the musculoskeletal disease is a mitochondrial-associated musculoskeletal disease. Skeletal muscle has high demands for ATP production. Given that mitochondria play a vital role in regulating cellular metabolism including ATP production, mitochondrial dysfunction is connected with musculoskeletal diseases. Musculoskeletal diseases include, but are not limited to tendinitis, carpal tunnel syndrome, osteoarthritis, rheumatoid arthritis (RA), and fibromyalgia bone fractures.

In some embodiments, the disease is a metabolic disease. In some embodiments, the metabolic disease is a mitochondrial-associated metabolic disease. Examples of metabolic diseases include, but are not limited to obesity, type 1 diabetes, and Niemann-Pick disease. As indicated above, mitochondria regulate cellular metabolism. Consequently, mitochondrial dysfunction can result in decreased energy production, which contributes to metabolic diseases.

E. Combining Biologically Active Synthetic Nanoparticle Constructs with Other Molecules

Some aspects of the present invention include combining the biologically active synthetic nanoparticle constructs with other molecules for a synergistic effect. For example, when using the biologically active synthetic nanoparticle constructs to treat cancer, the constructs can be combined with any cancer therapeutic. Examples of cancer therapeutics that can be combined with the biologically active synthetic nanoparticle constructs include, but are not limited to checkpoint inhibitors, chemotherapy, radiation, gene therapies, hormone therapies (e.g., aromatase inhibitors, selective estrogen receptor modulators (SERMs), Fulvestrant, androgen deprivation therapy, androgen blockers, etc.), and immunotherapies. In some cases, more than one cancer therapy can be administered in combination with the biologically active synthetic nanoparticle constructs.

In some embodiments, the biologically active synthetic nanoparticle constructs can be combined with a synthetic analog of coenzyme Q10, a nitric oxide donor (e.g., L-arginine), an NAD modulator (e.g., KL1333), cysteamine bitartrate (PROCYSBI®), or a ROS redox modulator (e.g., KH176). In further embodiments, the biologically active synthetic nanoparticle constructs can be combined with therapies that protect against redox perturbation or therapies that enhance peroxidase activity.

### Checkpoint Inhibitors

Examples of checkpoint inhibitors include, but are not limited to CTLA-4 inhibitors, PD-1 inhibitors, LAG-3 inhibitors, and PD-L1 inhibitors. PD-1 inhibitors include, but are not limited to pembrolizumab (KEYTRUDA®), nivolumab (OPDIVO®), dostarlimab (JEMPERLI®), and cemiplimab (LIBTAYO®). PD-L1 inhibitors include, but are not limited to atezolizumab (TECENTRIQ®), avelumab (BAVENCIO®), and durvalumab (IMFINZI®). CTLA-4 inhibitors include, but are not limited to iplimumab (YERVOY®) and tremelimumab (IMJUNO®). LAG-3 inhibitors include, but are not limited to relatimab.

### Chemotherapies

Examples of chemotherapies include, but are not limited to alkylating agents, antimetabolites, anti-tumor antibiotics, topoisomerase inhibitors, and mitotic inhibitors. Examples of alkylating agents include, but are not limited to Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, and Trabectedin. Examples of antimetabolites include, but are not limited to Azacitidine, 5-fluorouracil (5-FU), 6-mercaptopurine (6-MP), Capecitabine (XELODA®), Cladribine, Clofarabine, Cytarabine (Ara-C), Decitabine, Floxuridine, Fludarabine, Gemcitabine (GEMZAR®), Hydroxyurea, Methotrexate, Nelarabine, Pemetrexed (ALITMA®), Pralatrexate, Thioguanine, and Trifluridine/tipiracil combination. Examples of alkylating agents include, but are not limited to anthracyclines (e.g., Daunorubicin, Doxorubicin (ARIAMYCIN®), Doxorubicin liposomal, Epirubicin, Idarubicin, and Valrubicin) and non-anthracyclines (Bleomycin, Dactinomycin, Mitomycin-C, and Mitoxantrone). Examples of topoisomerase inhibitors include, but are not limited to topoisomerase I inhibitors (i.e., Irinotecan, Irinotecan liposomal, and Topotecan) and topoisomerase II inhibitors (Etoposide (VP-16), Mitoxantrone (also acts as an anti-tumor antibiotic), and Teniposide). Examples of mitotic inhibitors include, but are not limited to Cabazitaxel, Docetaxel, Nab-paclitaxel, Paclitaxel, Vinblastine, Vincristine, Vincristine liposomal, and Vinorelbine.

In the case of using the biologically active synthetic nanoparticle constructs to treat neurodegenerative diseases or disorders the constructs can be combined with an approved treatment for the neurodegenerative disease or disorder of interest. In some cases, the biologically active synthetic nanoparticle constructs can be combined with antioxidants (e.g., Idebenone).

In other embodiments, the biologically active synthetic nanoparticle constructs may further be combined with anti-inflammatory drugs. In further embodiments, the biologically active synthetic nanoparticle constructs can be combined with polypeptide dendrimer nanoparticles (See U.S. Pat. No. 11,306,326).

### F. Other Applications of Biologically Active Synthetic Particle Constructs

In some embodiments, the biologically active synthetic particle constructs can be used in combination with super resolution fluorescence microscopy (such as stimulated emission depletion (STED) microscopy, structured illumination microscopy (SIM), and stochastic optical reconstruction microscopy (STORM)/photoactivation localization microscopy (PALM)) to study transportation across the mitochondrial membrane (e.g., via the mitochondrial membrane transport protein). Examples of such proteins whose transportation across the mitochondrial membrane can be studied include mitochondrial fission and fusion proteins. In further embodiments, the biologically active synthetic particle constructs can be used in combination with single cell sequencing to study the mechanism of gene/redox regulation.

### Examples

### Example 1. Materials and Methods

This Examples details the materials and methods used in Example 2.

### Example 1A. Synthesis of Pyrrole-Imidazole Polyamide (PIP) Ligands

Light strand promoter (LSP)-targeting PIP and heavy strand promoter (HSP)-targeting PIP were synthesized by adapting the previously reported approach using solid-phase synthesis (Li et al. 2022, Zielonka et al. 2017, and Vafai et al. 2012). Briefly, PIPs were covalently assembled from individual units of Fmoc-Cha-OH, Fmoc-D-Arg (Pbf)-OH, Fmoc-N-methylimidazole-OH, or Fmoc-N-methylpyrrole-OH by presenting these units to N-methylpyrrole-coupled oxidme resin in a sequential manner. The acetyl group was coupled to the N-terminal of the assembly units using acetic anhydride, which resulted in the capping of the N-terminal. Afterward, the resins containing samples were digested by N, N-dimethylaminopropylamine under heating (55° C.) for three hours, and the solution was flowed into ether solvent, leading to the formation of solid substances. The precipitates were then harvested and vacuum dried. Samples were further purified using reverse-phase column chromatography (CombiFlash Rf, C18 RediSep Rf reverse-phase flash column from Teledyne Isco, Inc), as well as using a reverse-phase S2 HPLC (Jasco Engineering UV 275 coupled with a PU-2080 plus series system and a UV/Vis detector). A preparative column from the YMC-Pack Pro C18 series (150×20 mm) or Chemcobond (5-ODS-H, 4.6×150 mm) was used for the purification process. The mobile phase used in the purification is based on MeCN and H₂O supplemented with 0.1% TFA (trifluoracetic acid). Matrix-assisted laser desorption/ionization-time of flight (MALDI-TOF) mass spectrometry and HPLC (High-performance liquid chromatography) were used to characterize the amine-terminated LSP and HSP PIPs.

### Example 1B. Synthesis of Biologically Active Synthetic Nanoparticle Constructs and Control Nanoparticles

Glutathione (GSH)-functionalized gold nanoclusters were synthesized by adapting a previously reported approach (Chen et al. 2013). Glutathione as a linker is a short, zwitterionic with free carboxylic groups for conjugation. 100 mL 8 mM HAuCl₄ solution was heated to 90° C. in a three-neck round bottom flask under vigorous stirring. Then 100 mL of freshly prepared GSH solution (6 mM) was injected into the HAuCl₄ solution, followed by continuous stirring for 6 hours. The solution turned yellow and was then cooled down to room temperature. Afterward, the resulting nanocluster solution was dialyzed against ultrapure water three times and then concentrated to 3 mg/mL by evaporating water in a rotary evaporator at 60° C. for 3-4 hours. The concentration of the nanocluster solution was measured by entirely evaporating 1.0 mL nanocluster solution in an oven, followed by measuring the remaining powder mass. The nanocluster solution was stored at room temperature for one week and in the fridge for long-term storage. Conjugation of GSH-functionalized nanoclusters with PIP and MPP ligands was performed through 1-Ethyl-3-(3-dimethylaminopropyl) carbodiimide (EDC)/N-hydroxysuccinimide (NHS, Thermo Fisher Scientific) coupling. Briefly, the nanocluster solution was diluted to 1 mg/mL using 10 mM 2-(N-morpholino) ethanesulfonic buffer (MES buffer) with a final pH of around 6. For the synthesis of LSP-targeting biologically active synthetic nanoparticle constructs, 50 uL of 10 mM (16 mg/mL) LSP-NH₂ solution (in DMSO) and 50 uL of 10 mM MPP-NH₂ solution were mixed in an ice bath and then added into 1 mL of 1 mg/mL nanocluster solution under vortex, followed by shaking for 6 hours at room temperature. Biologically active synthetic nanoparticle constructs formed in the solution were then purified by filtration through an Amicon® ultrafilter membrane under centrifugation at 5000 rpm for one hour, and then the precipitates on the membrane were resuspended in PBS. The process was repeated 2-3 times to remove the unreacted ligands and the MES buffer thoroughly. Biologically active synthetic nanoparticle constructs, composed of varying ratios of LSP and MPP, were synthesized by adjusting the molar ratio between the amount of LSP-NH₂ ligands while keeping the concentration of MPP-NH₂ ligands constant. For example, a biologically active synthetic nanoparticle construct with LSP:MPP ratio of 4:1 is synthesized by adding 200 uL instead of 50 uL of 10 mM solution of LSP-NH₂ into the nanoclusters, while keeping the volume and concentration of MPP-NH₂ solution identical (50 uL). Nanoclusters conjugated with HSP and MPP were also synthesized using an identical approach, while replacing LSP-NH2 with HSP-NH₂ during the synthesis. In the size tuning experiment, 5 nm, 3 nm, and 2 nm nanoclusters were synthesized from the same reaction between HAuCl₄ and GSH, but at different concentrations of GSH solutions (2 mM, 4 mM, and 8 mM for 5 nm, 3 nm, and 2 nm, respectively). 10 nm GSH-conjugated gold nanoparticles were synthesized by ligand exchange using a commercialized 10 nm gold nanoparticle (Ted Pella). Briefly, 100 uL 100 mM GSH solution was added to 1.0 mL citrate-capped gold nanoparticles, followed by shaking at room temperature overnight. Then the GSH-conjugated gold nanoparticles were purified by an Amicon® filter membrane with identical procedures, as mentioned above.

### Example 1C. Nanoparticle and Biologically Active Synthetic Nanoparticle Construct Characterization

Nanoparticles and biologically active synthetic nanoparticle constructs were characterized by transmission electron microscope (TEM), Zeta sizer, Zeta potential, and UV-Vis (ultraviolet visible) absorption spectrum. In zeta potential measurement, the nanoparticles were suspended in an aqueous solution at a concentration of 50 ug/mL and then sonicated for 5 minutes before the measurement. Each experiment has at least 11 runs and 3 repeated measurements. For TEM imaging, nanoparticles were diluted to 10 μg/ml in ultrapure water, and the diluted solution was then drop-cast to TEM grids and vacuum-dried overnight. TEM imaging was performed at 80 kV on a Philips CM12 model coupled with AMT digital camera (model: XR111). The UV-Vis absorption and fluorescence spectrum of nanoparticles were measured in a quartz cuvette by Varian Cary 50® UV-Vis Spectrophotometer and Cary Eclipse Fluorescence Spectrometer from Agilent, respectively. To quantify the ligand conjugation to the biologically active synthetic nanoparticle constructs, two different ratios (2.5 and 10) of initial (Ri) PIP to MPP ligands were used for the conjugation to nanoclusters. Specifically, after the conjugation of PIP (10 ug equivalent) and MPP ligands (25 ug equivalent) to the nanoclusters (100 ug equivalent), potassium cyanide (KCN) was used to dissolve gold atoms in the biologically active synthetic nanoparticle constructs (100 uL with a concentration of 1 mg/mL, total of 100 ug), followed by extensive washing by water and re-suspension of hydrophobic PIP and MPP ligands in DMSO. The dissolved DMSO solution was then used for liquid chromatography mass spectroscopy (LC-MS, XevoR G2-XS Qtof, MeCN/water as solvent pair, retention time at 2.4 and 2.3 peak for PIP and MPP, respectively) analysis of concentrations of PIP and MPP ligands dissolved from biologically active synthetic nanoparticle constructs. From the LC-MS results, the amount of PIP (based on m/z peak area at 820, 14 ug/mL in 100 uL DMSO, or 1.40 ug) and MPP (based on m/z peak area at 713, 4 ug/mL in 100 uL DMSO, or 0.4 ug) ligands was calculated. Given the total amount of biologically active synthetic nanoparticle construct is around 100 ug, the weight percentages of PIP and MPP are 1.4% and 0.4%, respectively. According to literature 2 nm gold nanoclusters correspond to 144 gold atoms and 75 atoms on the surface, with a molecular weight of 28368. Therefore, the ratio between nanocluster, PIP, and MPP is (100/28368):(10/1640):(10/1426)=12.5:3:1.

### Example 1D. Potential Effects from Ligand Cleavage from Biologically Active Synthetic Nanoparticle Constructs

Since the seminal paper by Kim et al. 2010, the potential intracellular cleavage of thiol-gold bond been considered as a concern for cellular applications of gold nanoparticles. The gold-thiol bond is generally considered to be partially covalent and can be slowly dissolved at concentrated biothiol solutions. However, this would only partially compromise the ligand on the nanoparticle surfaces and the remaining ligands can still play a role in multivalency effects. This is evidenced by numerous studies on better gene manipulation efficiency in spherical nucleic acids as well as the NanoScript platform as compared to free ligands (Patel et al. 2014). If all ligands were dissolved in the cytoplasm then it would be surprising to still observe the advantages of gold nanomedicine in many drug/gene delivery applications. For thio-capped gold nanocluster specifically, there have also been a few studies where fluorescent dye was conjugated to nanoclusters for förster resonance energy transfer (FRET)-based monitoring of ligand localization inside cytoplasm, including in the same cell line used herein (HeLa cells) (Pyo et al. 2018). From their results, it is apparent that a significant amount of ligand remained on the surface of nanoclusters rather than fully dissolved in the cytoplasm. To provide evidence on this, LC-MS analysis on the free ligands derived from biologically active synthetic nanoparticle construct-treated cells was performed and the results preliminarily indicated an absence of degradation as well. In parallel, even partial dissolution of the ligands below the detection limit of LC-MS could still affect the overall performance of biologically active synthetic nanoparticle constructs. Therefore, one could use platinum-based nanoclusters, which supposedly have better affinity to thiol ligands (Qing et al. 2020). Alternatively, using carbene or selenol-based conjugation chemistry would be another option (Serva et al. 2015).

### Example 1E. Biologically Active Synthetic Nanoparticle Construct Ligand Stability After Cellular Uptake

To provide evidence on this point, an experiment was performed to analyze the existence of free ligand in cells treated with biologically active synthetic nanoparticle constructs for 24 hours. Specifically, biologically active synthetic nanoparticle constructs were administered to HeLa cells via incubation (5000 cells per well, 10 ug biologically active synthetic nanoparticle construct per well) and the media was not changed for 24 hours. Cells were then detached with trypsin, washed with 1×PBS (phosphate-buffered saline), and lysed using TRIzol™. DMSO was also added to ensure full dissolution of free ligands inside the cells, and the concentration was adjusted to be equivalent to the free biologically active synthetic nanoparticle construct ligand dissolution (by KCN) study. The same conditions (MeCN and water solvent pair and column conditions) were used for the analysis and the concentration was quantified by 820 m/z peak area. As a control, untreated cells were also harvested and analyzed using identical procedures. From these results, PIP ligand detachment from biologically active synthetic nanoparticle constructs was observed, but to a minimum level of 4.9% after 24 hours treatment when compared to total PIP ligand dissolved from KCN treatment of biologically active synthetic nanoparticle constructs. Consequently, these results suggest that a majority of ligands remain intact on biologically active synthetic nanoparticle constructs, and supports multivalency claims.

### Example 1F. Cell Culture, Biologically Active Synthetic Nanoparticle Construct Delivery, and Cell Imaging

HeLa cells were cultured and maintained in 10% fetal bovine serum (FBS, Thermo Fischer) of Dulbecco's Modified Eagle Medium (DMEM) basal media. Media was changed every other day, and the cells were passaged when they reached 100% confluency. For the biologically active synthetic nanoparticle construct, PIP molecule, and control nanoparticle delivery studies, cells were seeded at 20% confluency in a 24-well plate and allowed to adhere for 24 hours before the delivery experiment. For the delivery, biologically active synthetic nanoparticle constructs were directly suspended in serum-free OptiMEM® at a concentration of 10 μg/ml, then the cells in the 24-well plate were replaced with the biologically active synthetic nanoparticle construct OptiMEM® media suspension and incubated for 24 hours before the media was changed. The biologically active synthetic nanoparticle constructs are typically administered to cells in OptiMEM® for six hours followed by media change into growth media.

PIP molecules were first dissolved in DMSO at a concentration of 10 mM, then diluted into the OptiMEM® media at a concentration of 10 μM, and then the cell media was replaced. Note that PIP molecules are hydrophobic, and DMSO (dimethyl sulfoxide) has to be used for dissolving PIP molecules.

Forty-eight hours after the delivery, cells were imaged for ROS levels (via DCFDA) or analyzed for cell viability and mitochondrial gene expression. To image the cellular uptake and mitochondria targeting of the biologically active synthetic nanoparticle constructs, Nikon Ti series epifluorescence microscope and Zeiss LSM 800 confocal microscope were used. The visualization of biologically active synthetic nanoparticle constructs was achieved both by its intrinsic NIR fluorescence (excitation at 400 nm and emission at 650 nm) and the labeling of a Cy5-NHS dye to the biologically active synthetic nanoparticle constructs through the reaction between NHS ester and the amine group of glutathione ligands on the biologically active synthetic nanoparticle constructs. Fluorescent images were then processed using Nikon NIS Elements software and Zen Blue software.

To verify the reproducibility of the biologically active synthetic nanoparticle construct system, the same set of experiments were performed on human iPSC-derived neural stem cells (hiPSC-NSCs) (Yang et al. 2018 and Yang et al. 2022). hiPSC-NSC, hiPSC, THP-1 monocyte growth media was formulated from 1:1 DMEM/F12 and neurobasal media, with the addition of GlutaMAX™, B-27™, and bFGF (FGF-basic). In contrast to HeLa cells, the delivery of biologically active synthetic nanoparticle constructs to hiPSC-NSCs was performed using growth media instead of OptiMEM® at a concentration of 10 μg/ml. Cells in a 24-well plate were incubated for 48 hours before they were harvested for qRT-PCR analysis of ND6 gene expression. Detailed cell culture media is listed in Table 4.

TABLE 4
Media formulations and reagents used in cell culture.

| | hiPSC-NSCs | RenCells | THP-1 cells | iPSCs |
|---|---|---|---|---|
| Substrate | Laminin (Sigma Millipore | Laminin (Sigma | None | Matrigel |
| coating | CC095)/ Matrigel | Millipore CC095) | | (Corning) |
| | (Corning) | | | |
| Media | DMEM/F12 with | neural basal medium | RPMI1640 | PeproGrow ™ |
| | GlutaMAX ™, | (Gibco) and DMEM/F12 | with 10% FBS | hESC |
| | (Invitrogen), B-27 ™ | (Gibco) (50:50 ratio) | | (Embryonic |
| | Supplement (Invitrogen), | supplemented with 0.5% | | Stem Cell) |
| | N2 (Stem Cells), and | N-2 (Gibco), 0.5% B- | | Media |
| | 20 ng per mL bFGF | 27 ™, and 20 ng/ml | | |
| | (Invitrogen) | EGF and bFGF | | |
| | | (Fibroblast growth factor- | | |
| | | basic, PeproTech) | | |

Note
that biologically active synthetic nanoparticle construct-based suppression of ND6 gene would lead to the defects in Complex I in the mitochondria membrane which further leads to reduction of mitochondria function and increase ROS (reactive oxygen species) inside the cell.

### Example 1G. Mitochondria Co-localization Analysis

Biologically active synthetic nanoparticle constructs were directly suspended in serum-free OptiMEM® at a concentration of 10 μg/ml, then cells in a thin-glass bottom well plate were treated with the constructs by incubation for 24 hours before the media was changed. Then the cells were further treated with MitoTracker™ Green dye (Catalog #M7514) using protocols suggested by the vendor. Specifically, cell media with an equivalent concentration of 50 nM MitoTracker™ Green was prepared. Cells were treated with MitoTracker™ Green for 20 minutes in the incubator followed by 1×PBS wash. Then a removable live-cell imaging chamber was placed under a Zeiss LSM 800 confocal microscope for the imaging of biologically active synthetic nanoparticle construct and MitoTracker™ co-localization. After obtaining the images, the co-localization efficiency was calculated through a standard Pearson test in MatLab software package (codes are available). A high co-localization co-efficiency (calculated based on Pearson score) and minimal nanocluster fluorescence in the nucleus region indicate the efficient mitochondrial transportation of biologically active synthetic nanoparticle constructs.

### Example 1H. qRT-PCR Analysis of Mitochondrial Gene Expression

To lyse the cells and analyze mitochondrial gene expression, cells were treated using the RNeasy Mini kit from QIAGEN. Reverse transcription of the total RNA was then performed using 200 ng of RNA by a qPCR kit following the manufacturer's recommendations. To analyze the expression of mitochondrial RNA, a LightCycler® kit from Roche in combination with a SYBR q-PCR mix kit from ThermoFisher were used for the converted DNA. The ratio between ND6 expression levels and MT-16S expression levels was used to quantify the efficiency of mitochondrial gene suppression due to the biologically active synthetic nanoparticle constructs and PIP molecules. Sequences of the PCR primers are shown in Table 5.

TABLE 5
The primer sequence for the
genes which are analyzed.

| | | SEQ | | SEQ |
|---|---|---|---|---|
| | | ID | | ID |
| Gene | Forward Primer | NO: | Reverse Primer | NO: |
| MT-16S | 5′-ACTTTGCA | 10 | 5′-GCTATCACC | 11 |
| | AGGAGAGCCAA | | AGGCTCGGTAG | |
| | A | | | |
| ND6 | 5′-GGGTTAGC | 12 | 5′-GATCCTCCC | 13 |
| | GATGGAGGTAG | | GAATCAACCCT | |
| | G | | | |

### Example 1I. Cell Viability and ROS Assay

Cell viability was quantified by a PrestoBlue® Cell Viability Assay from Thermo Fisher (Catalog Number A13261). The Presto Blue® reagent was diluted 10 times into the media, and cells were incubated at 37° C. for 20 minutes before absorbance at 570 nm was measured by a plate reader. Cell viability was normalized to untreated control cells. For the ROS assay, live cells were treated with 10 UM dichloro-dihydro-fluorescein diacetate (DCFH-DA) and incubated at 37° C. for 20 minutes, then washed 3 times with 1×PBS. Cells were then imaged using a Nikon microscope under the FITC channel. Light exposure was kept constant across different conditions, and ROS levels were quantified by measuring intracellular fluorescent intensities using the automatic functions of Nikon Element Air software.

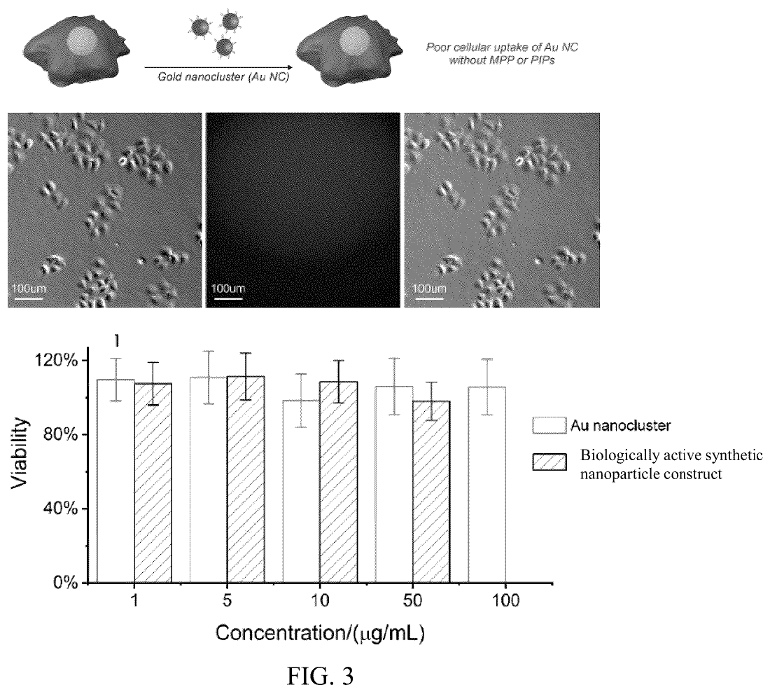

Note that HeLa cells were treated with Au nanocluster and a biologically active synthetic nanoparticle construct for 48 hours before being analyzed by PrestoBlue™ Cell Viability Assay (FIG. 3).

### Example 2. Investigation of Performance of Biologically Active Synthetic Nanoparticle Constructs

### Example 2A. Generation of Biologically Active Synthetic Nanoparticle Construct and its Suitability for Mitochondria-Based Applications

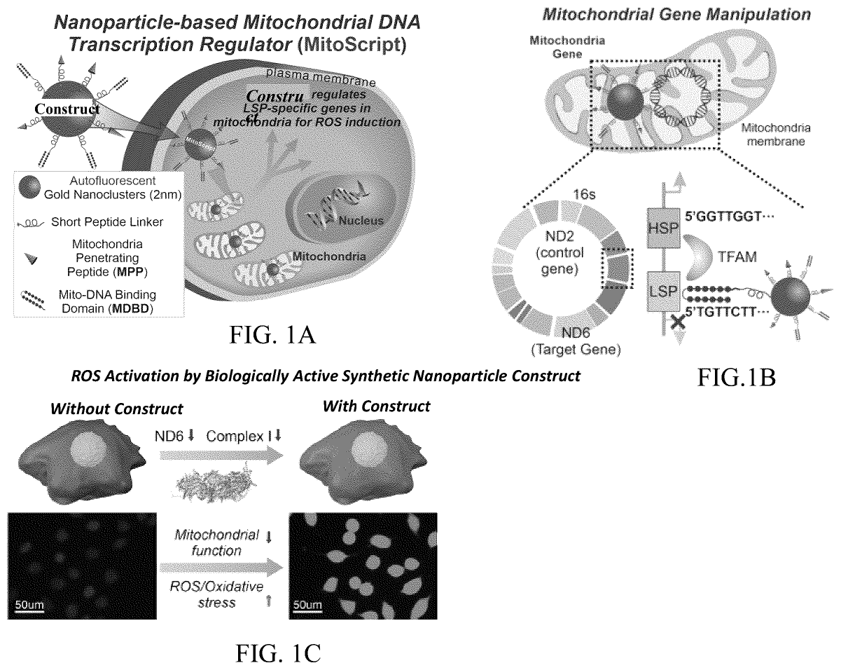

To overcome the challenges associated with the regulation of mitochondrial gene expression and redox manipulation, a biomimetic biologically active synthetic nanoparticle construct-based approach that facilitate overcoming the cell membrane and mitochondrial barriers were developed. Biologically active synthetic nanoparticle constructs were generated and characterized as discussed above in Example 1. As shown in FIG. 1A, the biologically active synthetic nanoparticle construct partially recapitulates the multi-domain structures of natural mitochondria transcription factors (TFs), by grafting multiple mitochondria penetration domains (MPP peptide) and DNA binding domains (PIP oligomers) onto a single nanocluster, which effectively translocate the DNA binding domains into the mitochondria instead of the nucleus.

The construct has sizes (2-3 nm) smaller than natural transcription factors, but it adds additional unique functions, such as an innate near-infrared red (NIR) fluorescence for mitochondria tracking. Additionally, a multivalency effect resulting from the higher densities of surface conjugation sites can be used to assemble MPP and DNA binding domains.

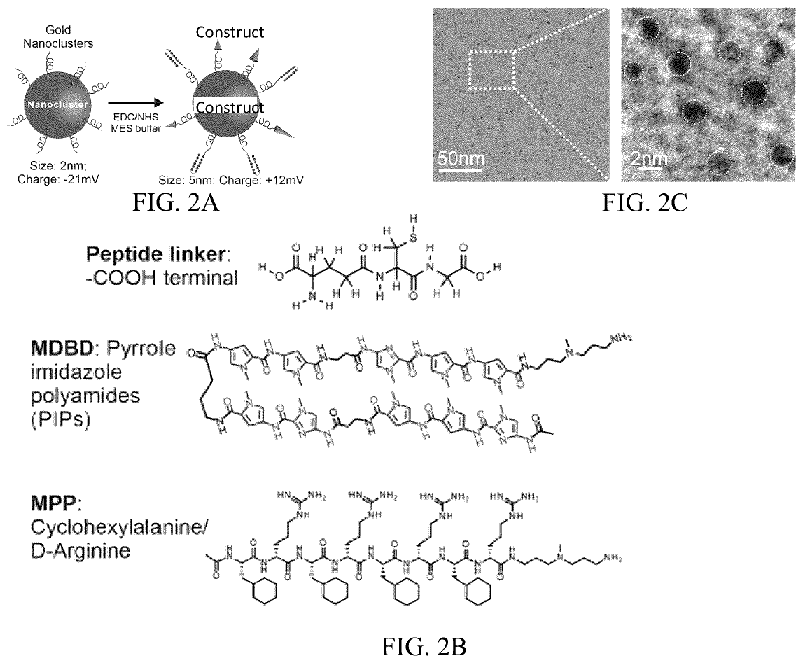

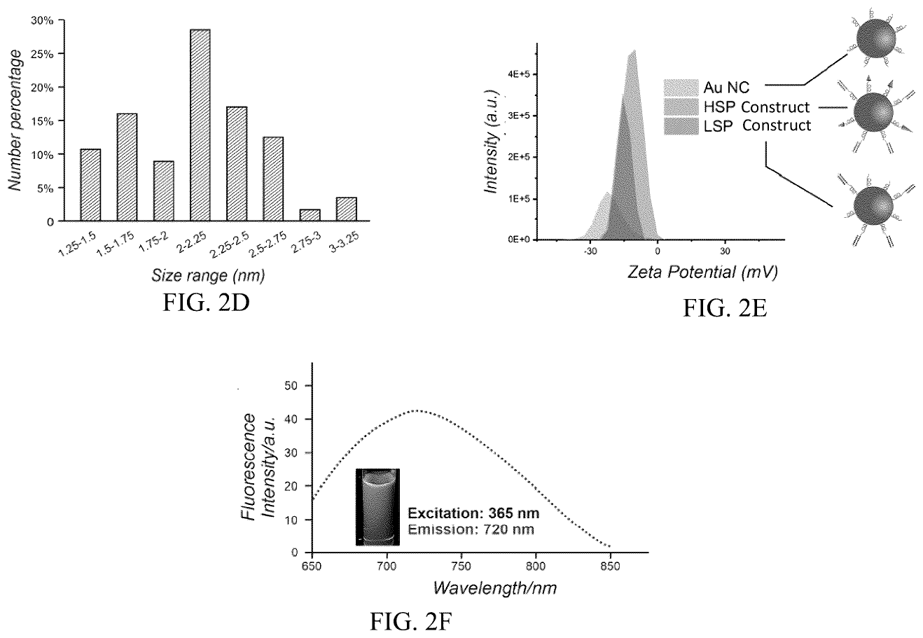

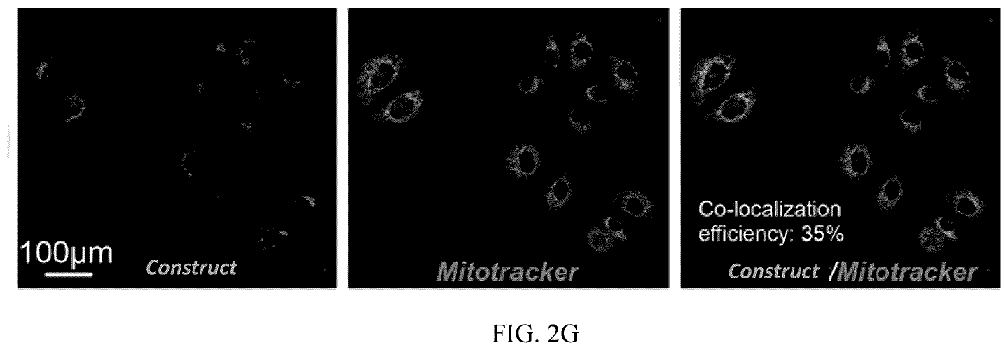

To demonstrate that a multivalency effect can be used to assemble MPP and DNA binding domains, fluorescent ultrasmall gold nanoclusters were first synthesized from a facile redox reaction between gold (III) chloride and glutathione (GSH) peptide (FIG. 2A). In this reaction, the thiol group in GSH serves both as a reducing agent and capping agent for restricted crystal growth, yielding 2-3 nm gold nanoclusters with 100-200 carboxylic groups on the surfaces of each particle. Liquid chromatography mass spectroscopy (LCMS) was used to calculate approximately 32% coverage in terms of ligand conjugation on the biologically active synthetic nanoparticle constructs. The ligands were also found to be stable for at least 24 hours after cellular uptake, thereby validating the conjugation strategy.

In parallel, amine-functionalized hairpin polyamide was synthesized with amino acid sequences of ImPyPyβImPy-γ-PyPyβImPyPy-βDp-NH₂ was synthesized using solid-phase synthesis (FIG. 2B). In hairpin polyamides, N methylimidazole (Im) and N-methylpyrrole (Py) amino acids are known to selectively bind complementary G-C and A-T motifs on both nuclear and mitochondrial DNA. Specifically, the amino acid sequence is designed to bind to the light strand promoter (LSP), which can alter gene expression on the light strand of mitochondrial DNA.

Similarly, MPP with an optimal ratio of hydrophobic cyclohexylalanine (Cha) and positive d-arginine (Arg) that allows for robust targeting of the mitochondrial membrane was also synthesized with a residual amine group. An equal amount of amine-functionalized PIP and MPP were conjugated to carboxylic groups on gold nanoclusters and assembled into biologically active synthetic nanoparticle constructs. Considering its ultrasmall size, innate NIR fluoresce, multivalent surfaces, excellent cellular uptake, low batch-to-batch variation, and high biocompatibility compared to the nanocluster or PIP molecule alone, the biologically active synthetic nanoparticle constructs could be suitable for mitochondria-based applications (FIGS. 2C-2F, 3, and 4A-4E)

### Example 2B. Cellular Uptake of Biologically Active Synthetic Nanoparticle Constructs

To achieve mitochondrial transcription regulation, efficient transportation across cellular membranes must occur. Efficient delivery of biologically active synthetic nanoparticle constructs to HeLa cells was studied using a fluorescence microscope based on the intrinsic fluorescent properties of the nanoclusters at the core of the biologically active synthetic nanoparticle constructs (FIG. 2F). Note that PIP molecules are hydrophobic and DMSO must be used to dissolve such molecules. In contrast, the constructs do not require toxic solvents for cell delivery. Thus, avoiding the use of organic solvents or viral transfection vectors, enhances biocompatibility and potentially reduces immunogenicity. Notably, the biologically active synthetic nanoparticle constructs were found to be stable in most physiological buffers, including phosphate-buffered saline (PBS) and cell growth media, which could be attributed to the dense hydrophilic GSH linker on its surfaces (FIG. 2E). This contrasts with previously reported PIP molecules, which also inhibit mitochondrial gene expression, but are poorly soluble in water or any physiologically relevant buffers).

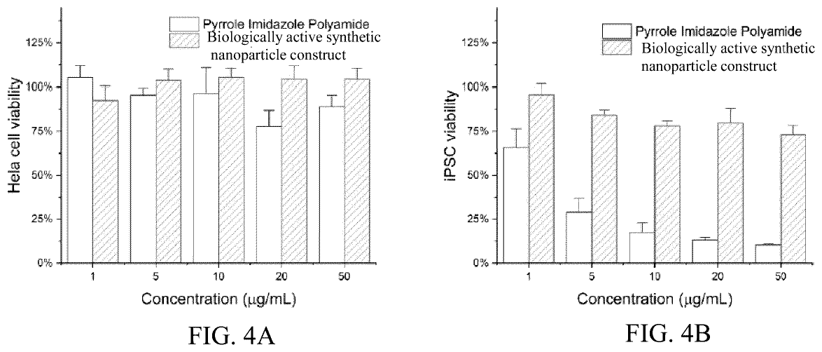

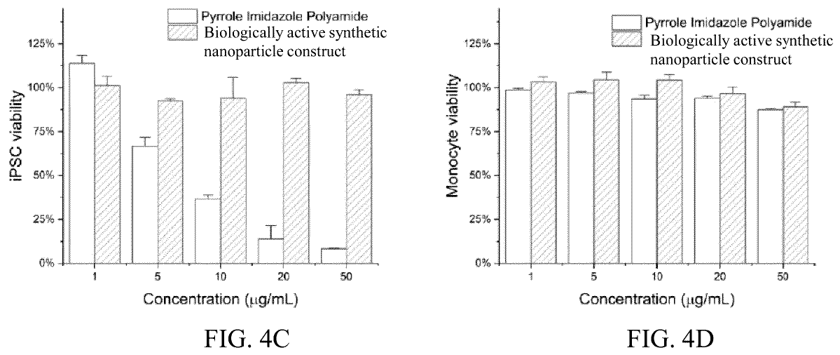

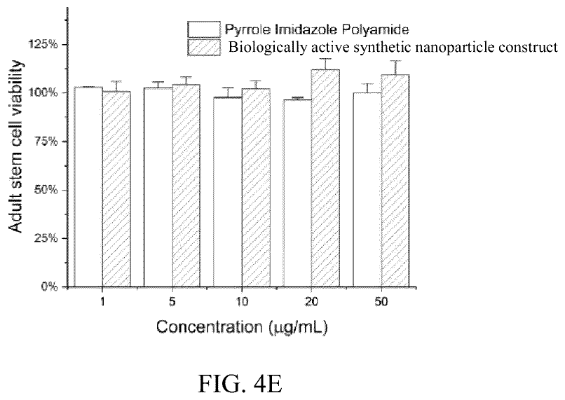

Overall, even at a relatively low concentration (10 μg/mL, or 100 nM), the biologically active synthetic nanoparticle constructs were rapidly (within 24 hours) uptaken by nearly all cells, with an over 96% delivery efficiency. Also, because of its high biocompatibility and avoidance of using organic solvents during the delivery, the construct at both low (10 μg/mL) and high concentrations (100 μg/mL) was found to be non-toxic (FIGS. 3 and 4A-4E), not only to the standard HeLa cell line, but also to more delicate human induced pluripotent stem cell-derived neural stem cells, as well as human monocytes. Moreover, the minimal difference in cell viability between HeLa cells administered Au nanoclusters vs. biologically active synthetic nanoparticle constructs suggests excellent biocompatibility of the biologically active synthetic nanoparticle constructs. The high efficiency of cellular uptake and increased biocompatibility of the biologically active synthetic nanoparticle constructs represent clear advantages over conventional DNA-binding motifs, including free PIP molecules.

### Example 2C. Delivery of Biologically Active Synthetic Nanoparticle Constructs to Mitochondria

Furthermore, biologically active synthetic nanoparticle constructs that were efficiently uptaken by cells effectively targeted the mitochondria, as demonstrated by confocal microscope-based live-cell imaging (FIG. 2G). Specifically, mitochondria in Hela cells were stained with tetramethylrhodamine isocyanate (TRITC) labeled MitoTracker®, and to make biologically active synthetic nanoparticle constructs visible using confocal microscopy, nanoclusters were conjugated with N-hydroxyl succinimide (NHS) conjugated cyanine 5 (Cy5) dye using the residual amine groups in the GSH linker. A high co-localization (approximately 35% from Pearson score) of biologically active synthetic nanoparticle construct fluorescence with TRITC signals in the live-cell imaging experiment suggests selective delivery of biologically active synthetic nanoparticle constructs into the mitochondria versus the cytosol (note that DAPI nuclei staining was not used because the fluorescence overlaps with the nanoparticle constructs). Further optimization can be achieved by enhancing the MPP to PIP ratio. Likewise, as the delivery of DNA-binding motifs into the nucleus has also been widely used to manipulate nuclear gene transcription, it is crucial to ensure a minimal presence of biologically active synthetic nanoparticle constructs in the nucleus.

Additionally, minimal biologically active synthetic nanoparticle construct fluorescence signals in nuclear regions was observed in the confocal microscope images (FIG. 2G). Such efficient and selective delivery of biologically active synthetic nanoparticle constructs into the mitochondria are prerequisites of mitochondrial gene manipulation and could be attributed to MPPs on biologically active synthetic nanoparticle constructs. Although DNA-binding motifs have also been conjugated with MPPs for selective delivery into mitochondria, high delivery efficiency at nanomolar concentrations was achieved without any organic solvents or viral vectors. This result is consistent with previous literature in spherical nucleic acids, where oligonucleotides conjugated to nanoparticles result in better transfection efficiency than free oligonucleotides, not only because of the alteration of cellular uptake mechanism, but also reduced enzymatic degradation of DNA-binding motifs and multivalent binding of target DNAs on nanoparticles.

### Example 2D. Ability of Biologically Active Synthetic Nanoparticle Constructs to Regulate Gene Transcription in Mitochondria

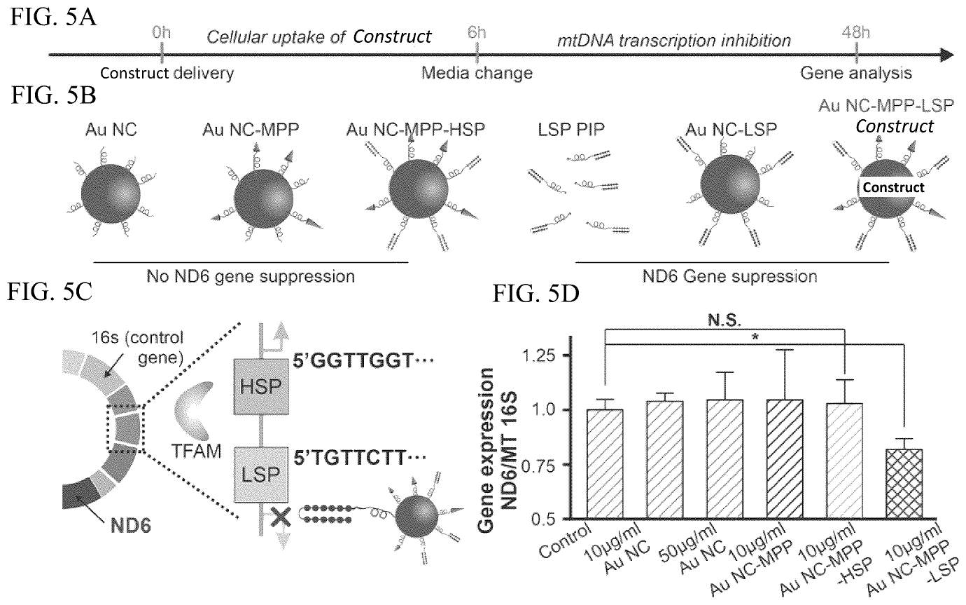

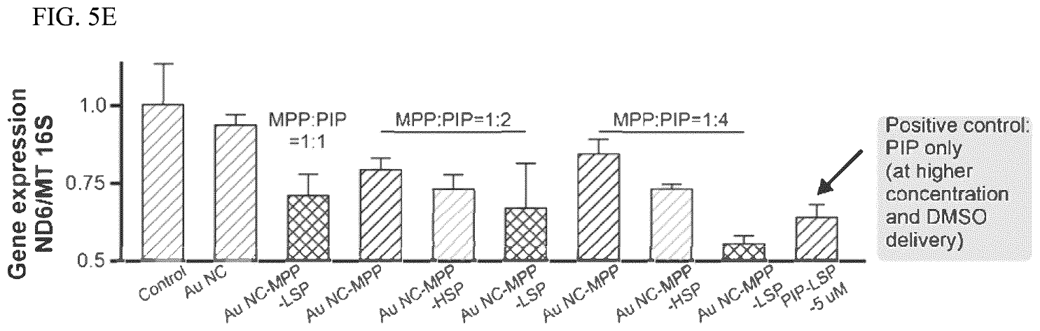

Next, the ability of biologically active synthetic nanoparticle constructs to regulate gene transcription in mitochondria was evaluated (FIG. 5). Biologically active synthetic nanoparticle constructs are typically assembled from multiple DNA-binding domains (PIP molecules), MPP domains, and a small-sized nanocluster as their cores. Biologically active synthetic nanoparticle construct with LSP-targeting PIP ligand only was not included in this study because it is well established that MPP is essential for biomolecular delivery into mitochondria. In view of the foregoing results, it was hypothesized that i) both DNA-binding domains and MPPs are essential for the selective manipulation of mitochondrial gene transcription; ii) multiple domains assembled on nanoclusters would have multivalency effects on gene expression; and iii) the small size of nanoclusters is crucial for efficient mitochondrial gene manipulation (FIGS. 5A and 5B).

To prove this, mitochondrial genes were isolated and analyzed 48 hours after delivery of biologically active synthetic nanoparticle constructs that target the light strand promoter (LSP) region using 16S (mitochondrially encoded 16S RNA) as a baseline gene (FIG. 5C). Nanoclusters conjugated with LSP-targeting PIPs only, with MPPs only, or with both domains were synthesized and tested as controls. The PIP was engineered to target the HSP region in the mitochondrial genome under identical mass concentrations (FIG. 5D). Nanoclusters alone or nanoclusters conjugated with MPP only did not exhibit any regulatory effects on mitochondrial gene expression. Among all groups, only biologically active synthetic nanoparticle constructs showed significant suppression of ND6, which is a direct indicator of regulation of LSP-associated mitochondrial gene manipulation, according to previous reports. This directly justified the design of the multi-domain structures of the biologically active synthetic nanoparticle construct platform. Furthermore, as positive controls, PIPs that target LSP were also administered at varying concentrations to Hela cells. As expected, high concentrations of (10 μM) solutions of PIPs transfected using dimethyl sulfoxide (DMSO) (2%) resulted in significant suppression of ND6 gene expression, to a similar level from a low concentration (100 nM) of biologically active synthetic nanoparticle construct treatment (FIG. 5E). However, delivery of PIPs requires DMSO which is toxic at high concentrations. When concentrations of PIPs were lowered to 5 μM their effects on gene expression decreased, despite that their concentration was still an order higher than biologically active synthetic nanoparticle constructs. This is again consistent with the literature on nanoparticle-based nuclear gene transcription regulation that the multivalency of DNA-binding motifs can facilitate the recognition and inhibition of target genes during transcription (Thaner et al. 2020). PIP molecule alone at a high concentration also induced suppression of ND6 gene expression, but their delivery requires DMSO which is toxic at high concentrations.

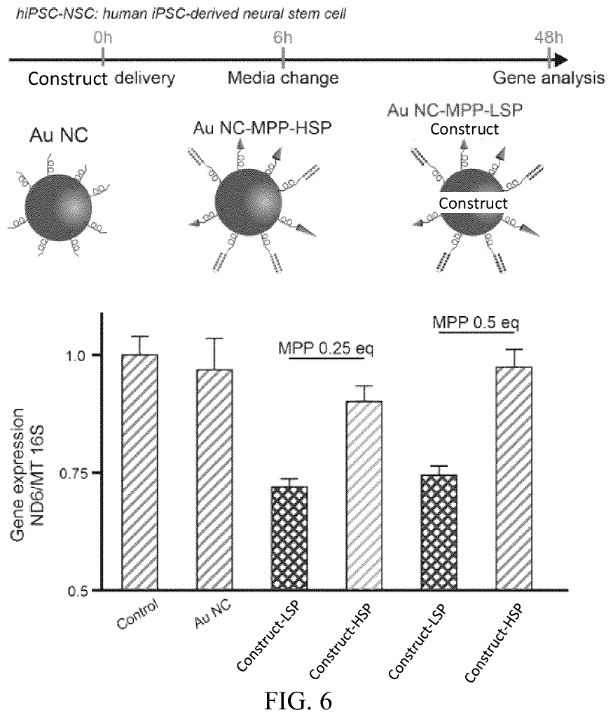

Remarkably, biologically active synthetic nanoparticle construct-based modulation of mitochondrial gene expression was also successfully reproduced in a human iPSC-NSC line. Only the biologically active synthetic nanoparticle constructs induced suppression of ND6 genes after 48 hours of biologically active synthetic nanoparticle construct treatment, while nanocluster alone or nanocluster conjugated with MPP alone did not show any clear effect on the ND6 gene expression (FIG. 6). This strongly supports biologically active synthetic nanoparticle constructs as a platform for mitochondrial gene expression in applications other than the model HeLa cell line.

### Example 2E. Role of the Size of Nanoclusters in Biologically Active Synthetic Nanoparticle Construct in Gene Regulation

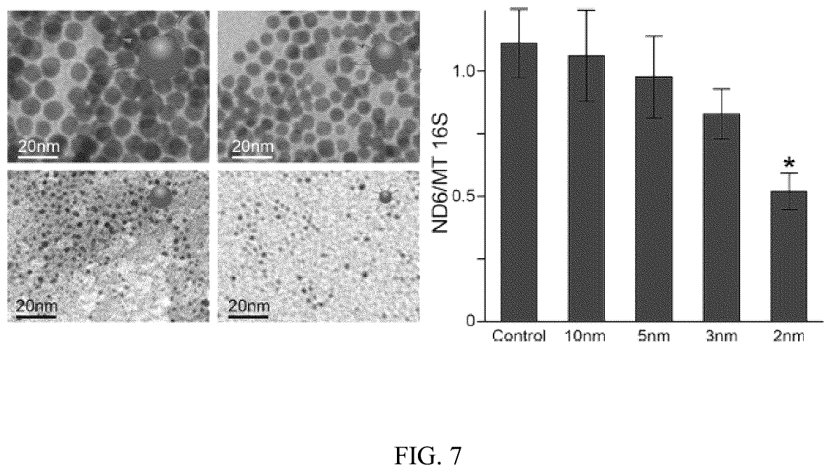

In addition, to study whether the size of nanoclusters in the biologically active synthetic nanoparticle constructs plays an essential role in gene regulation, nanoparticle constructs were synthesized using gold nanocluster/nanoparticles with varying sizes from 2 nm, 3 nm, 5 nm, and 10 nm (FIG. 7). As shown in FIG. 7, the graph on the right panel illustrates qRT-PCR analysis of mitochondrial ND6 gene expression after HeLa cells treated with biologically active synthetic nanoparticle constructs at varying sizes (n=3 biological replicates, *P<0.05 by one-way ANOVA with Tukey post-hoc analysis). While 2 and 3 nm gold nanoclusters were synthesized directly from GSH using similar protocols, 5 and 10 nm gold nanoparticles were synthesized from the citrate-based reduction of HAuCl₄, followed by ligand exchange with GSH, and conjugation with MPPs and PIPs through carbodiimide cross-linker. By doing so, similar surfaces across different gold nanoclusters/nanoparticles were assumed. Gene analysis of HeLa cells treated by biologically active synthetic nanoparticle constructs assembled from varying sizes revealed a clear trend of size-dependent effect on mitochondrial gene manipulation (FIG. 7). Specifically, a decrease in the nanoparticle sizes resulted in a more robust suppression of mitochondrial gene transcription. When sizes reach 10 nm, no effects from the MPP and PIP-conjugated nanoparticles on mitochondrial gene transcription were observed. This result is well-aligned with previous reports on protein transportation across the mitochondrial membrane, which unlike the nuclear membrane, lacks large pores that allow for the transportation of high-molecular-weight proteins. Taken together, by engineering the multi-domain structures and sizes, the biologically active synthetic nanoparticle construct platform was optimized, and its robust regulation of mitochondrial gene transcription in vitro was verified.

### Example 2F. Effect of Biologically Active Synthetic Nanoparticle Construct-based Gene Regulation on Cellular Activities

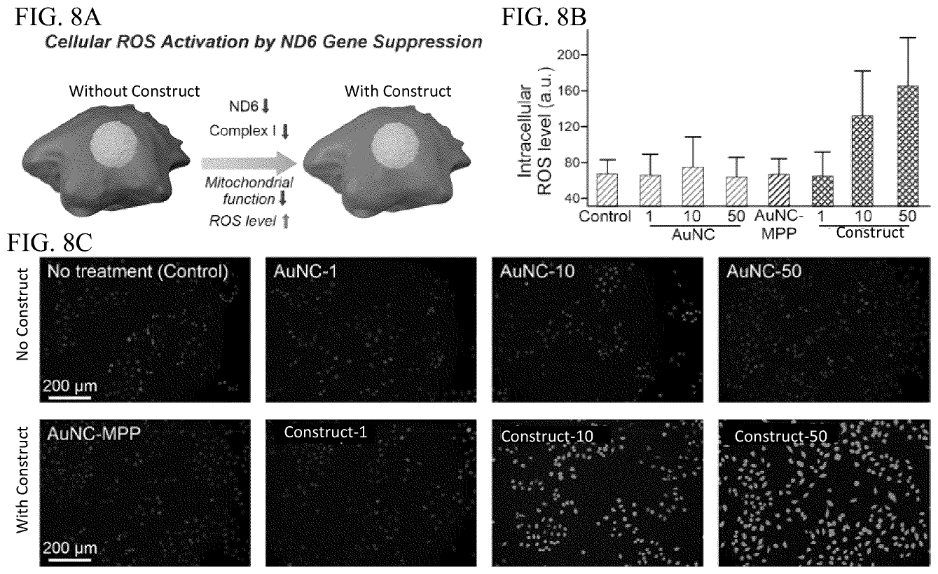

Selective and reliable manipulation of mitochondrial gene transcription can enable the regulation of energy production and redox balancing, which are of utmost importance for cancer migration, muscle contraction, and neuron death. To confirm whether biologically active synthetic nanoparticle construct-based gene regulation can alter cellular activities, biologically active synthetic nanoparticle construct-induced redox manipulation was studied as a proof-of-concept. ND6 is a key subunit of NADH dehydrogenase in the electron transport chain involved in ATP production. Suppression or mutation of ND6 genes in mitochondria results in ROS production (FIG. 8A). As such, it was hypothesized that the exemplary biologically active synthetic nanoparticle constructs that target LSP and suppress the ND6 gene could also manipulate cellular redox by inhibiting NADH dehydrogenase.

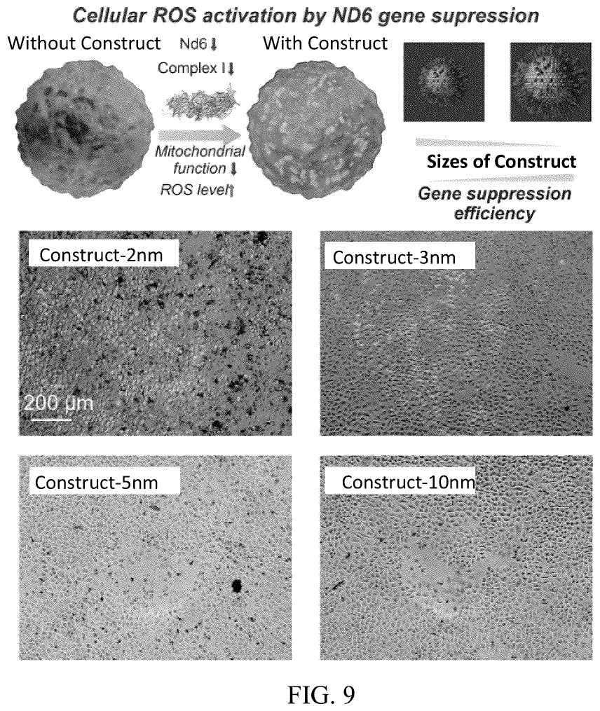

In order to test this hypothesis, HeLa cells were treated with varying concentrations of the biologically active synthetic nanoparticle constructs and investigated ROS production using a standard 2′-7′-Dichlorodihydrofluorescein diacetate (DCFH-DA)-based assay 48 hours after nanoparticle construct treatment (FIGS. 8B and 8C). As controls, nanoclusters with equivalent concentrations were also administered to the cells, and analysis was performed under identical conditions. This hypothesis was directly supported by observing a concentration-dependent increase of ROS levels in biologically active synthetic nanoparticle construct-treated HeLa cells, indicated by the increased intensities of fluorescent signal, while minimal changes were observed by bare nanoclusters treatment across all concentrations (FIGS. 8B and 8C). Strikingly, biologically active synthetic nanoparticle constructs assembled from different-sized gold nanoclusters/nanoparticles can also show an apparent decrease in ROS production when sizes were increased, which provides additional support for the observation in the size-dependent study on mitochondrial gene regulation (FIG. 9). Thus, the data indicates that biologically active synthetic nanoparticle constructs-based regulation of mitochondrial gene transcription can modify cellular functions, which can improve knowledge and treatment of mitochondrial gene-related diseases and disorders.

### CONCLUSION

In summary, inspired by the multi-domain structure of natural transcription factors, an ultrasmall nanoparticle-based platform was designed and synthesized an ultrasmall nanoparticle-based platform to manipulate mitochondrial gene transcription effectively. Given that there have been only a few methods to regulate the mitochondrial genome, the biologically active synthetic nanoparticle constructs offer a promising alternative that provides several benefits. These benefits include intrinsic NIR fluorescence for cellular tracking, multivalency effects for more efficient gene manipulation, and the avoidance of toxic organic solvents and immunogenic viral vectors.

Notably, the biologically active synthetic nanoparticle constructs were difficult to develop since there must be a balance among cellular uptake, gene regulation, and mitochondrial penetration. Moreover, glutathione was used as a linker, which is difficult for non-experts to design and synthesize. Lastly, the optimal ratio among different ligands (MPPs and DNA-binding motifs) is different than that of NanoScript (See U.S. Pat. Nos. 10,100,332 and 11,306,326). By engineering the versatile nanoparticle construct platform, it was demonstrated that the biologically active synthetic nanoparticle constructs alter cellular redox states by regulating mitochondrial gene transcription, which is important for various biological applications.

It will be appreciated by those skilled in the art that changes could be made to the embodiments described above without departing from the broad inventive concept thereof. It is understood, therefore, that this invention is not limited to the particular embodiments or examples disclosed, but it is intended to cover modification that are within the spirit and scope of the present invention as defined by the appended claims.

Sequence Listing
ODN1
SEQ ID NO: 1
5′-GCGAACAGTCACCC
ODN2
SEQ ID NO: 2
5′-GGGTGACTGTTCGC
ODN3
SEQ ID NO: 3
5′-GCTCCGAACCACAG
ODN4
SEQ ID NO: 4
5′-CTGTGGTTCGGAGC
Repression Domain
SEQ ID NO: 5
WRPW
Repression Domain
SEQ ID NO: 6
RLITLADHICQIITQDFAR
Repression Domain
SEQ ID NO: 7
QINDLYSTDRPESAEAPDLOSWELR
Repression Domain
SEQ ID NO: 8
ELQKSIGHKPEPTEEWELIKTVTEAHV
Repression Domain
SEQ ID NO: 9
STPSSKTKDLGHNDKKSS
Forward Primer for MT-16S
SEQ ID NO: 10
ACTTTGCAAGGAGAGCCAAA
Reverse Primer for MT-16S
SEQ ID NO: 11
GCTATCACCAGGCTCGGTAG
Forward Primer for ND6
SEQ ID NO: 12
GGGTTAGCGATGGAGGTAGG
Reverse Primer for ND6
SEQ ID NO: 13
GATCCTCCCGAATCAACCCT
AD peptide domain
SEQ ID NO: 14
SGLMDLDFDDLADSGLMDLDFDDLADSGC

### REFERENCES

1. Chen, T.; Hu, Y.; Cen, Y.; Chu, X.; Lu, Y., A Dual-Emission Fluorescent Nanocomplex of Gold-Cluster-Decorated Silica Particles for Live Cell Imaging of Highly Reactive Oxygen Species. Journal of the American Chemical Society 2013, 135 (31), 11595-11602.
2. Kim, B.; Han, G.; Toley, B. J.; Kim, C.-k.; Rotello, V. M.; Forbes, N. S., Tuning payload delivery in tumour cylindroids using gold nanoparticles. Nature Nanotechnology 2010, 5 (6), 465-472.
3. Li, X.; Straub, J.; Medeiros, T. C.; Mehra, C.; den Brave, F.; Peker, E.; Atanassov, I.; Stillger, K.; Michaelis, J. B.; Burbridge, E., Mitochondria shed their outer membrane in response to infection-induced stress. Science 2022, 375 (6577), eabi4343. Patel, S.; Jung, D.; Yin, P. T.; Carlton, P.; Yamamoto, M.; Bando, T.; Sugiyama, H.; Lee, K.-B., NanoScript: a nanoparticle-based artificial transcription factor for effective gene regulation. ACS nano 2014, 8 (9), 8959-8967.
4. Pyo, K.; Ly, N. H.; Han, S. M.; Hatshan, M. b.; Abuhagr, A.; Wiederrecht, G.; Joo, S.-W.; Ramakrishna, G.; Lee, D., Unique Energy Transfer in Fluorescein-Conjugated Au22 Nanoclusters Leading to 160-Fold pH-Contrasting Photoluminescence. The Journal of Physical Chemistry Letters 2018, 9 (18), 5303-5310.
5. Qing, Z.; Luo, G.; Xing, S.; Zou, Z.; Lei, Y.; Liu, J.; Yang, R., Pt—S Bond-Mediated Nanoflares for High-Fidelity Intracellular Applications by Avoiding Thiol Cleavage. Angewandte Chemie International Edition 2020, 59 (33), 14044-14048.
6. Serva, S.; Lagunavičius, A. n., Direct Conjugation of Peptides and 5-Hydroxymethylcytosine in DNA. Bioconjugate Chemistry 2015, 26 (6), 1008-1012. Thaner, R. V.; Eryazici, I.; Macfarlane, R. J.; Brown, K. A.; Lee, B.; Nguyen, S. T.; Mirkin, C. A., The Significance of Multivalent Bonding Motifs and “Bond Order” in DNA-Directed Nanoparticle Crystallization. In Spherical Nucleic Acids, Jenny Stanford Publishing: 2020; pp 851-862.
7. Yang, L.; Chueng, S.-T. D.; Li, Y.; Patel, M.; Rathnam, C.; Dey, G.; Wang, L.; Cai, L.; Lee, K.-B., A biodegradable hybrid inorganic nanoscaffold for advanced stem cell therapy. Nature Communications 2018, 9 (1), 3147.
8. Yang, L.; Conley, B. M.; Yoon, J.; Rathnam, C.; Pongkulapa, T.; Conklin, B.; Hou, Y.; Lee, K.-B., High-Content Screening and Analysis of Stem Cell-Derived Neural Interfaces Using a Combinatorial Nanotechnology and Machine Learning Approach. Research 2022, 2022, 9784273.
9. Yang, L; Rathnam, C. l Hidaka, T.; Hou, Y.; Conklin, Brandon.; Pandian, G.; Sugiyama, H.; Lee, K-B., Nanoparticle-Based Artificial Mitochondrial DNA Transcription Regulator: MitoScript. Nano Letters. 2023, 23 (5), 2046-2055.
10. Zielonka, J.; Joseph, J.; Sikora, A.; Hardy, M.; Ouari, O.; Vasquez-Vivar, J.; Cheng, G.; Lopez, M.; Kalyanaraman, B., Mitochondria-Targeted Triphenylphosphonium-Based Compounds: Syntheses, Mechanisms of Action, and Therapeutic and Diagnostic Applications. Chemical Reviews 2017, 117 (15), 10043-10120.
11. Vafai, S. B.; Mootha, V. K., Mitochondrial disorders as windows into an ancient organelle. Nature 2012, 491 (7424), 374-383.

What is claimed is:

1. A biologically active synthetic nanoparticle construct comprising:
i. a nanocluster that allows for conjugation of multiple biomolecular ligands,
ii. a plurality of linkers,
iii. a plurality of single copies of mtDNA-binding domains constructed to bind selectively to the light strand promoter or heavy strand promoter of a target mitochondrial gene and suppress gene expression; and
iv. a plurality of mitochondrial penetrating linkers,
wherein the nanoparticle selectively targets the mitochondria of a cell.

2. The biologically active synthetic nanoparticle construct of claim 1, wherein the nanoparticle size is between 1 to 3 nm.

3. The biologically active synthetic nanoparticle construct of claim 1, wherein the nanocluster is gold, platinum, silver, aluminum, copper or alloys thereof, or a chalcogen.

4. The biologically active synthetic nanoparticle construct of claim 3, wherein the nanocluster is gold.

5. The biologically active synthetic nanoparticle construct of claim 1, wherein the nanocluster exhibits innate near-infrared red (NIR) fluorescence.

6. The biologically active synthetic nanoparticle construct of claim 1, wherein the linkers are selected from the group consisting of hydrophilic glutathione (GSH) peptides, proteins, polyethylene glycol, polysaccharides, lipids, alkynes, alkanes, polyamines, carbene-terminated ligands, and zwitterionic polymers.

7. The biologically active synthetic nanoparticle construct of claim 6, wherein the linkers are GSH peptides.

8. The biologically active synthetic nanoparticle construct of claim 1, wherein the mitochondrial penetrating linkers are selected from the group consisting of triphenylphosphonium, phenylalanine- and cyclohexyl alanine-based mitochondrial penetrating peptides.

9. The biologically active synthetic nanoparticle construct of claim 1, wherein the construct further comprises a plurality of repression domains.

10. The biologically active synthetic nanoparticle construct of claim 1, wherein the mtDNA-binding domains are pyrrole-imidazole polyamide (PIP) ligands.

11. The biologically active synthetic nanoparticle construct of claim 10, wherein the PIP ligands are LSP-NH₂ PIP ligands or HSP-NH₂ PIP ligands.

12. The biologically active synthetic nanoparticle construct of claim 11, wherein the conjugation ratio between mitochondrial penetrating linkers and PIPs is between 1:1 and 1:4.

13. The biologically active synthetic nanoparticle construct of claim 1, wherein said nanoparticle inhibits mitochondrial gene expression.

14. The biologically active synthetic nanoparticle construct of claim 13, wherein the mitochondrial gene is selected from the group consisting of ATPF06, ATPF08, COI, COII, COIII, CYTB, ND1, ND2, ND3, ND4, ND4l, ND5, and ND6.

15. The biologically active synthetic nanoparticle construct of claim 13, wherein inhibiting mitochondrial gene expression increases ROS levels.

16. A method of inhibiting mitochondrial gene expression, wherein the method comprises administering a biologically active synthetic nanoparticle construct comprising:
i. a nanocluster that allows for conjugation of multiple biomolecular ligands,
ii. a plurality of linkers,
iii. a plurality of single copies of mtDNA-binding domains constructed to bind selectively to the light strand promoter or heavy strand promoter of a target mitochondrial gene and suppress gene expression; and
iv. a plurality of mitochondrial penetrating linkers,
wherein the nanoparticle selectively targets the mitochondria of a cell.

17. The method of claim 16, wherein said mitochondrial gene is selected from the group consisting of ATPF06, ATPF08, COI, COII, COIII, CYTB, ND1, ND2, ND3, ND4, ND4l, ND5, and ND6.

18. The method of claim 16, wherein the nanoparticle size is between 1 to 3 nm.

19. The method of claim 16, wherein the nanocluster is gold, platinum, silver, aluminum, copper or alloys thereof, or a chalcogen.

20. The method of claim 19, wherein the nanocluster is gold.
